(19) United States
(12) Patent Application Publication
(10) Pub. No.: US 2013/0117885 A1
(43) Pub. Date: May 9, 2013

(54) Novel Microrna Precursor and Methods of Use for Regulation of Target Gene Expression

(71) Applicants: Yuwen Wang, Apex, NC (US); Lawrence Winfield Talton, Cary, NC (US); Aaron Wiig, Durham, NC (US); Peifeng Ren, Cary, NC (US)

(72) Inventors: Yuwen Wang, Apex, NC (US); Lawrence Winfield Talton, Cary, NC (US); Aaron Wiig, Durham, NC (US); Peifeng Ren, Cary, NC (US)

(73) Assignee: BASF Plant Science Company GmbH, Ludwigshafen (DE)

(21) Appl. No.: 13/807,412

(22) PCT Filed: Jun. 28, 2011
(86) PCT No.: PCT/IB2011/052837

§ 371 Date: Dec. 28, 2012

Related U.S. Application Data
(60) Provisional application No. 61/359,864, filed on Jun. 30, 2010.

(51) Int. Cl. A01H 1/06

(52) U.S. Cl. CPC A01H 1/06
USPC 800/279; 435/419; 435/320.1; 536/23.1; 536/24.5; 800/278; 800/298

(57) ABSTRACT

Methods for modulating target gene expression in plants by expression of recombinant microRNA precursors are provided. Uses of the said recombinant microRNA precursors for the control of nematodes, in particular the control of soybean cyst nematodes are also provided. Methods for introduction of genetic material into plants that are susceptible to nematodes in order to increase resistance to nematodes are further provided.

### ABSTRACT

The present invention is in the field of plant molecular biology and provides methods for modulating target gene expression in plants by expression of a recombinant microRNA precursor. The invention is also directed to the use of such recombinant microRNA precursors for the control of nematodes, in particular the control of soybean cyst nematodes. The invention also relates to the introduction of genetic material into plants that are susceptible to nematodes in order to increase resistance to nematodes.

### DESCRIPTION OF THE INVENTION

Gene expression in plants is a highly controlled mechanism, regulated in all steps involved. Those are for example, accessibility of the genomic DNA for the transcriptional machinery or regulation of stability of the messenger. In the last years it has been shown, that stability and accessibility of RNA molecules such as messenger RNA is highly regulated by small interfering RNAs (siRNAs) such as, for example microRNAs, ta-siRNAs and others.

MicroRNAs have emerged as evolutionarily conserved, RNA-based regulators of gene expression in animals and plants. MicroRNAs (approx. 18 to 25 nt) arise from larger precursors, pre-miRNAs, with a stem loop structure that are transcribed from non-protein-coding genes.

Plant microRNAs known so far repress expression of a high number of genes which function in developmental processes, indicating that microRNA-based regulation is integral to pathways governing growth and development. Gene expression-repressing plant microRNAs usually contain near-perfect complementarity with target sites, which occur most commonly in protein-coding regions of mRNAs (Llave C et al. (2002) Science 297, 2053-2056; Rhoades M W et al. (2002) Cell 110, 513-520). As a result, in plants most gene expression-repressing plant microRNAs function to guide target RNA cleavage (Jones-Rhoades M W and Bartel D P (2004) Mol. Cell 14, 787-799; Kasschau K D et al. (2003) Dev. Cell 4, 205-217).

Various publications describe the function of microRNAs and their use as tool for downregulation of target gene expression by overexpression of endogenous or recombinant microRNAs in plants. Ossowski et al (2008) are giving an overview on methods for gene silencing using artificial siRNAs such as microRNAs.

Schwab et al (2006) have shown in Arabidopsis that not all microRNA precursors work equally well for silencing when engineered for repression of target genes. For example precursors MIR319a and MIR172a engineered for targeting the same target gene show different degree of downregulation of the target gene, the MIR319a being more efficient in silencing the respective target gene.

Moreover, a microRNA precursor well suited for efficient target gene silencing in one species may not work equally well in another specie (Alvarez J P et al., 2006 Plant Cell 18: 1134-1151).

It was therefore one aim of the invention at hand to provide further microRNA precursors that can be used as efficient tools for target gene silencing in plants by delivering small regulatory RNAs such as microRNAs, ta-siRNAs, siRNAs, activating RNAs and the like. It was another aim of the invention to provide microRNA precursors that can be used as efficient tools for target gene silencing in plants of the genus Glycine, preferably in Glycine max.

Such recombinant microRNA precursors are for example an efficient tool for modulation of genes involved in the interaction between plant and pathogens, for example nematodes thereby preventing infection and increasing pathogen resistance of plants, such as Soy.

Nematodes are microscopic roundworms that feed on the roots, leaves and stems of more than 2,000 row crops, vegetables, fruits, and ornamental plants, causing an estimated $100 billion crop loss worldwide. A variety of parasitic nematode species infect crop plants, including root-knot nematodes (RKN), cyst- and lesion-forming nematodes. Root-knot nematodes, which are characterized by causing root gall formation at feeding sites, have a relatively broad host range and are therefore pathogenic on a large number of crop species. The cyst- and lesion-forming nematode species have a more limited host range, but still cause considerable losses in susceptible crops.

Pathogenic nematodes are present throughout the United States, with the greatest concentrations occurring in the warm, humid regions of the South and West and in sandy soils. Soybean cyst nematode (Heterodera glycines), the most serious pest of soybean plants, was first discovered in the United States in North Carolina in 1954. Some areas are so heavily infested by soybean cyst nematode (SCN) that soybean production is no longer economically possible without control measures. Although soybean is the major economic crop attacked by SCN, SCN parasitizes some fifty hosts in total, including field crops, vegetables, ornamentals, and weeds.

Signs of nematode damage include stunting and yellowing of leaves, and wilting of the plants during hot periods. However, nematode infestation can cause significant yield losses without any obvious above-ground disease symptoms. The primary causes of yield reduction are due to root damage underground. Roots infected by SCN are dwarfed or stunted. Nematode infestation also can decrease the number of nitrogen-fixing nodules on the roots, and may make the roots more susceptible to attacks by other soil-borne plant pathogens.

The nematode life cycle has three major stages: egg, juvenile, and adult. The life cycle varies between species of nematodes. For example, the SCN life cycle can usually be completed in 24 to 30 days under optimum conditions whereas other species can take as long as a year, or longer, to complete the life cycle. When temperature and moisture levels become favorable in the spring, worm-shaped juveniles hatch from eggs in the soil. Only nematodes in the juvenile developmental stage are capable of infecting soybean roots.

The life cycle of SCN has been the subject of many studies, and as such are a useful example for understanding the nematode life cycle. After penetrating soybean roots, SCN juveniles move through the root until they contact vascular tissue, at which time they stop migrating and begin to feed. With a stylet, the nematode injects secretions that modify certain root cells and transform them into specialized feeding sites. The root cells are morphologically transformed into large multinucleate syncytia (or giant cells in the case of RKN), which are used as a source of nutrients for the nematodes. The actively feeding nematodes thus steal essential nutrients from the plant resulting in yield loss. As female nematodes feed, they swell and eventually become so large that their bodies break through the root tissue and are exposed on the surface of the root.

After a period of feeding, male SCN nematodes, which are not swollen as adults, migrate out of the root into the soil and fertilize the enlarged adult females. The males then die, while the females remain attached to the root system and continue to feed. The eggs in the swollen females begin developing, initially in a mass or egg sac outside the body, and then later within the nematode body cavity. Eventually the entire adult female body cavity is filled with eggs, and the nematode dies. It is the egg-filled body of the dead female that is referred to as the cyst. Cysts eventually dislodge and are found free in the soil. The walls of the cyst become very tough, providing excellent protection for the approximately 200 to 400 eggs contained within. SCN eggs survive within the cyst until proper hatching conditions occur. Although many of the eggs may hatch within the first year, many also will survive within the protective cysts for several years. A nematode can move through the soil only a few inches per year on its own power. However, nematode infestation can be spread substantial distances in a variety of ways. Anything that can move infested soil is capable of spreading the infestation, including farm machinery, vehicles and tools, wind, water, animals, and farm workers. Seed sized particles of soil often contaminate harvested seed. Consequently, nematode infestation can be spread when contaminated seed from infested fields is planted in non-infested fields. There is even evidence that certain nematode species can be spread by birds. Only some of these causes can be prevented.

Traditional practices for managing nematode infestation include: maintaining proper soil nutrients and soil pH levels in nematode-infested land; controlling other plant diseases, as well as insect and weed pests; using sanitation practices such as plowing, planting, and cultivating of nematode-infested fields only after working non-infested fields; cleaning equipment thoroughly with high pressure water or steam after working in infested fields; not using seed grown on infested land for planting non-infested fields unless the seed has been properly cleaned; rotating infested fields and alternating host crops with non-host crops; using nematicides; and planting resistant plant varieties.

Methods have been proposed for the genetic transformation of plants in order to confer increased resistance to plant parasitic nematodes. U.S. Pat. Nos. 5,589,622 and 5,824,876 are directed to the identification of plant genes expressed specifically in or adjacent to the feeding site of the plant after attachment by the nematode. The promoters of these plant target genes can then be used to direct the specific expression of detrimental proteins or enzymes, or the expression of antisense RNA to the target gene or to general cellular genes. The plant promoters may also be used to confer nematode resistance specifically at the feeding site by transforming the plant with a construct comprising the promoter of the plant target gene linked to a gene whose product induces lethality in the nematode after ingestion.

Although there have been numerous efforts to use gene suppression methods to control plant parasitic nematodes, to date no transgenic nematode-resistant plant has been deregulated in any country. Accordingly, there continues to be a need to identify safe and effective compositions and methods for the controlling plant parasitic nematodes using RNAi or recombinant microRNA precursors, and for the production of plants having increased resistance to plant parasitic nematodes.

### DETAILED DESCRIPTION OF THE INVENTION

A first embodiment of the invention is directed to an isolated nucleic acid molecule comprising a nucleic acid molecule comprised in the group consisting of

I) a nucleic acid molecule represented by SEQ ID NO: 1, 3 and 10 and

II) a nucleic acid molecule having at least 100, preferably at least 143 for example 150, more preferably at least 195 for example 200, more preferably at least 250, even more preferably 355, most preferably 500 consecutive base pairs of a sequence described by SEQ ID NO: 1 wherein the at least 100 by comprise base pair 151 to 250 of SEQ ID NO: 1, the at least 143 by for example 150 comprise base pair 139 to 281 of SEQ ID NO: 1, the at least 195 by for example 200 by or 250 by comprise base pair 127 to 321 of SEQ ID NO: 1 and the at least 355 by for example 500 by comprise base pair 10 to 363 of SED ID NO: 1, respectively and

III) a nucleic acid molecule having an identity of at least 70%, preferably at least 80%, more preferably at least 85% or 90%, even more preferably at least 95%, 97%, 98% most preferably 99% over a sequence of at least 100, preferably at least 143 for example 150, more preferably at least 195 for example 200, more preferably at least 250, more preferably 355, even more preferably 500, most preferably 793 consecutive base pairs of a sequence described by SEQ ID NO: 1 wherein the at least 100 by comprise base pair 151 to 250 of SEQ ID NO: 1, the at least 143 by for example 150 comprise base pair 139 to 281 of SEQ ID NO: 1, the at least 195 by for example 200 by or 250 by comprise base pair 127 to 321 of SEQ ID NO: 1 and the at least 355 by for example 500 by comprise base pair 10 to 363 of SED ID NO: 1, respectively and

IV) a nucleic acid molecule of at least 100, preferably 150, more preferably 200, even more preferably 250, most preferably 500 by hybridizing under stringent, preferably high stringent, more preferably very high stringent conditions with a nucleic acid molecule of at least 100, preferably at least 143 for example 150, more preferably at least 195 for example 200, more preferably at least 250, even more preferably 355, most preferably 500 consecutive base pairs of a sequence described by SEQ ID NO: 1 wherein the at least 100 by comprise base pair 151 to 250 of SEQ ID NO: 1, the at least 143 by for example 150 comprise base pair 139 to 281 of SEQ ID NO: 1, the at least 195 by for example 200 by or 250 by comprise base pair 127 to 321 of SEQ ID NO: 1 and the at least 355 by for example 500 by comprise base pair 10 to 363 of SED ID NO: 1, respectively and

V) a nucleic acid molecule able to form or forming a secondary structure homologous, preferably identical to the secondary structure formed by SEQ ID NO: 1 and

VI) the complement of any of the sequences as defined in I) to V).

The nucleic acid molecule represented by SEQ ID NO: 1 or 10 encodes a novel plant microRNA precursor derived from Glycine max. The nucleic acid molecule represented by SEQ ID NO: 3 represents the genomic clone of the respective microRNA precursor gene represented by SEQ ID NO: 1 or 10. Such precursor molecules, once transcribed into RNA fold into specific secondary structures comprising stem-loop structures, also described as hairpin structures, wherein the stems may comprise bulges of non complementary basepairs or basepairs inserted in a stem on only one side of the stem. The precursor molecule folded into its respective secondary structure is recognized by the plant cell machinery which is then processing the precursor molecule and releasing the microRNA molecule sequence comprised in the respective precursor molecule. A fragment of SEQ ID NO: 1 comprising at least the said hairpin structure represented by base pair 139 to 281 of SEQ ID NO: 1 represents a functional fragment of the precursor microRNA molecule and is also comprised in the invention at hand.

The structure of the microRNA may for example be predicted by the use of nucleic acid secondary structure prediction software for example CentroidFold (Michiaki Hamada, et al (2009)), CONTRAfold (Do et al (2006)), KineFold (Xayaphoummine et al (2005)), Mfold (Zuker and Stiegler (1981)), Pknots (Rivas and Eddy (1999)), PknotsRG (Reeder et al (2007)), RNAfold (Hofacker et al (1994)), RNAshapes (Giegerich et al (2004)), RNAstructure (Mathews et al (2004)), Sfold (Ding et al (2004)) or UNAFold (Markham and Zuker (2008)).

The hairpin structure comprised in SEQ ID NO: 1, 3 and 10 comprising the microRNA sequence as depicted in FIG. 1 comprising base pair 139 to 281 of SEQ ID NO: 1 is one embodiment of the invention. This structure is recognized by the plant cell machinery and processed to release the microRNA comprised in said secondary structure of base pair 139 to 281 of SEQ ID NO: 1. The microRNA is located between base pair 225 and 235, the microRNA star is located between base pair 159 and 179 of SEQ ID NO: 1.

The secondary structure has been predicted using the bioinformatics tool Mfold (Zuker (2003)), using the following settings: RNA is defined as linear, folding temperature is fixed at 37° C., the ionic conditions are set as 1M NaCl, no divalent ions, percent suboptimality is set to 5, upper bound on the number of computed foldings is set to 50, the window parameter is default, maximum interior/bulge loop size is set to 30, the maximum asymmetry of an interior/bulge loop is set to 30, the maximum distance between paired bases is not limited. The skilled person is aware that with using other parameters or another program the secondary structure may be differently predicted. The hairpin structure of the nucleic acid molecule of the invention as defined above under V) may be described starting at the 5′ end of the sequence between base pair 139 and 281 of SEQ ID NO: 1 as comprising as follows:

8 bp stem, 1 bp bulge, 5 bp stem, 1 bp mismatch11 bp stem, 2 bp mismatch, 6 bp stem, 2 bp mismatch, 5 bp stem, 4 bp bulge, 2 bp stem, 2 bp bulge, 6 bp stem, 1 bp bulge, 3 bp stem, 9 bp loop, 3 bp stem, 2 bp bulge, 6 bp stem, 1 bp bulge, 2 bp stem, 2 bp bulge, 5 bp stem, 2 bp mismatch, 6 bp stem, 2 bp mismatch, 11 bp stem, 1 bp mismatch, 5 bp stem, 2 bp bulge, 4 bp stem, 6 bp loop, 4 bp stem, 3 bp bulge, 8 bp stem.

The functional nucleic acid molecule as defined above under V) may in one embodiment of the invention have variable sizes of the loops, the bulges and/or the stems. In addition, the number of mismatches and bulges may be varied. In one embodiment of the invention, the secondary structure as depicted in FIG. 1 may comprise at least 20, preferably at least 15, more preferably at least 10, even more preferably at least 5, most preferably 4, 3, 2 or 1 additional base pairs compared to base pair 139 and 281 of SEQ ID NO: 1. The secondary structure may in another embodiment of the invention comprise 20 or less, preferably 15 or less, more preferably 10 or less, even more preferably 5 or less, most preferably 4, 3, 2 or 1 fewer base pairs compared to base pair 139 and 281 of SEQ ID NO: 1.

For example, the size of the first loop may vary between 4 and 14 bp, preferably 5 to 13, preferably 6 to 12, more preferably 7 to 11, most preferably 8 to 10, wherein the boundary values are included.

In a further embodiment of the invention the size of the second loop may vary between 2 and 10 bp, preferably 3 to 9, preferably 4 to 9, more preferably 5 to 8, most preferably 5 to 7, wherein the boundary values are included. The size of one or both of the loops of the secondary structure as shown in FIG. 1 may vary.

The size of any of the stems of the secondary structure of the invention may be increased by for example 5, preferably 4, more preferably 3, even more preferably 2 most preferably 1 base pairs. Up to 10 further mismatches, for example 9 or 8 or 7, preferably 6 or 5 or 4, more preferably 3, even more preferably 2, most preferably 1 mismatches may be introduced in the secondary structure of the microRNA precursor molecule of the invention. The number of mismatches may in another embodiment of the invention be reduced to zero, preferably to 1, more preferably to 2, even more preferably to 3, most preferably to 4 mismatches.

Moreover, the number of bulges in the secondary structure may be increased or decreased by 2, preferably 1 bulge. The size of the bulges may be increased by up to 6 bases, for example 5 bases, preferably 4, more preferably 3, even more preferably 2, most preferably 1 base. The size of the bulges may also be decreased by 2, preferably 1 base.

The thermodynamic value of the structure of the invention as defined above under V) under the defined conditions is dG=-60.80.

The skilled person is aware, that the thermodynamic value might differ from this value without a loss of function of the respective secondary structure, hence the secondary structure of said microRNA precursor molecule would still be recognized by the plant cell and processed thereby releasing the microRNA molecule comprised in said microRNA precursor molecule. Hence in one embodiment of the invention, the dG value of the secondary structure of the invention may be between 40 and 80, preferably between 45 and 75, more preferably between 50 and 70, even more preferably between 55 and 65, most preferably between 57 or 58 or 59 and 64 or 63 or 62, wherein the boundary values are included.

The dG value of the hairpin structures comprised in the nucleic acid molecules represented by the sequences as defined in II) to VI) above when calculated with the same program and same conditions may deviate from the dG value calculated for the molecule represented by base pair 139 to 281 of SEQ ID NO: 1 as shown in FIG. 1 by 50% or less, preferably 30% or less, more preferably 25% or less, even more preferably 10% or less. In a most preferred embodiment, the dG value of the hairpin structures comprised in the nucleic acid molecules represented by the sequences as defined in II) to VI) above equals the dG value of the hairpin structure comprised in base pair 139 to 281 of SEQ ID NO: 1.

It is a further embodiment of the invention that in the isolated nucleic acid molecule as defined above

a) by 225-245 of SEQ ID NO:1 or the corresponding basepairs of the nucleic acid molecules as defined in above in II) to VI) are replaced by a nucleic acid sequence comprising at least 20 or 21 or 22 bp, preferably 21 bp, or a multitude thereof complementary to a target gene and

b) by 159-179 of SEQ NO: 1 or the corresponding basepairs of the nucleic acid molecules as defined above in II) to VI) are replaced by a nucleic acid sequence comprising at least 20 or 21 or 22 bp, preferably 21 bp, or a multitude thereof complementary to a) and wherein a) and b) and the nucleic acid molecule separating a) and b) are able to from a stem loop structure.

The basepairs as defined above under a) and b) represent the microRNA and the microRNA star molecules comprised in the microRNA precursor molecules as defined above. A double stranded molecule comprising both the microRNA and microRNA star molecule is released from the precursor molecule in the plant cell upon processing of said precursor molecule.

The microRNA star sequence may be completely complementary to the microRNA sequence or may comprise 1 or more, for example 1, or 2 or more, for example 2, or 3 or more, for example 3, or 4 or more, for example 4 mismatches when compared to the microRNA sequence.

In one embodiment, the isolated nucleic acid molecule comprises between 1 or more of the microRNA and microRNA star molecules. In a preferred embodiment, the isolated nucleic acid molecule comprises between 1 and 50 of the microRNA and microRNA star molecules. In a more preferred embodiment, the nucleic acid molecule comprises between 1 and 10, even more preferred between 1 and 5, in a most preferred embodiment it comprises 1 or 2, preferably 1 microRNA and microRNA star molecule.

“Corresponding basepairs” as used above means the basepairs comprised in the respective nucleic acid molecules represented by the sequences as defined above under II) to VI) that represent the microRNA and microRNA star molecule respectively. A skilled person is well aware how such corresponding basepairs may be identified. They may for example be identified by aligning SEQ ID NO: 1 with the sequences as defined above in II) to VI). The sequences aligned with by 159-179 and by 225-245 of SEQ ID NO: 1 respectively represent the respective corresponding basepairs.

The skilled person is aware on methods for design and synthesis of microRNA functional in a plant cell for modulating target gene expression.

The isolated nucleic acid as defined above, wherein by 225-245 of SEQ ID NO: 1 or the corresponding basepairs of the nucleic acid molecules as defined above in II) to VI) are replaced by a sequence selected from the group consisting of

a. a nucleic acid molecule represented by any of SEQ ID NO: 18, 19, 20 and/or 21 or a multitude thereof or

b. a nucleic acid molecule having at least 15 or 16, preferably 17, more preferably 18, even more preferably 19, most preferably 20 consecutive base pairs of a sequence described by any of SEQ ID NO: 18, 19, 20 and/or 21or a multitude thereof or

c. a nucleic acid molecule having an identity of at least 70%, preferably at least 75%, more preferably at least 80%, more preferably 90%, even more preferably 95% most preferably 97, 98 or 99% to the entire sequence of any of SEQ ID NO: 18, 19, 20 and/or 21or a multitude thereof or

d. a nucleic acid molecule comprising 5 mismatches, preferably 4 mismatches, preferably 3 mismatches, more preferably 2 mismatches, most preferably one mismatch when compared to any of SEQ ID NO: 18, 19, 20 and/or 21 or a multitude thereof,

e. a nucleic acid molecule hybridizing under high stringent conditions with a nucleic acid molecule described by any of SEQ ID NO: 18, 19, 20 and/or 21or a multitude thereof is a further embodiment of the invention.

A plant expression construct comprising the isolated nucleic acid molecule as defined in above is a further subject of the invention.

A plant expression construct as used herein means a nucleic acid molecule capable of directing expression of a particular nucleotide sequence in an appropriate part of a plant or plant cell, comprising a promoter functional in said part of a plant or plant cell into which it will be introduced, operatively linked to the isolated nucleic acid molecule of the invention which is—optionally—operatively linked to one or more termination signals.

Preferred promoters functionally or, as used synonymously herein, operatively linked to the isolated nucleic acid molecule of the invention are constitutive promoters, inducible promoters, preferably pathogen inducible promoters such as nematode inducible promoters, tissue specific, preferably root specific or nematode feeding site specific promoters and/or developmental specific promoters.

The invention is in addition directed to a plant expression vector comprising the isolated nucleic acid molecule as defined above or the expression construct as defined above. Preferably the plant expression vector is a viral vector, a plasmid vector or a binary vector.

A plant, plant cell or plant seed comprising the isolated nucleic acid molecule as defined above or the expression construct as defined in above or the plant expression vector as defined above is a further embodiment of the invention. Preferably, said expression construct or expression vector are inserted (at least in part) into the genome of the plant cell or plant. Another embodiment of the invention relates to transformed seed of the plant of the invention.

The present invention provides nucleic acids, transgenic plants, and methods to overcome or alleviate nematode infestation of valuable agricultural crops such as soybeans and potatoes. The nucleic acids of the invention are capable of decreasing expression of plant target genes by providing recombinant microRNA precursors.

In another embodiment, the invention provides a nematode resistant transgenic plant capable of expressing at least one a recombinant microRNA precursor of the invention, wherein the microRNA derived from said recombinant microRNA precursor inhibits expression of the target gene in the plant root or in the nematode and confers resistance of nematode infection.

One additional embodiment of the invention is a method for modulating, compared to a respective reference plant, expression of a target gene in a plant or part thereof comprising the steps of
a) functionally linking
   i. at least one regulatory nucleic acid molecule functional in a plant with
   ii. a recombinant microRNA precursor molecule heterologous to i) which is cleavable in a plant cell to produce said at least one regulatory nucleic acid molecule and
b) introducing this nucleic acid molecule into a plant or part thereof

wherein the sequence of the at least one regulatory nucleic acid molecule is heterologous to the sequence of said microRNA precursor molecule and

wherein the sequence of the at least one regulatory nucleic acid molecule is binding to at least one target sequence in the plant and

wherein the microRNA precursor molecule is selected from a nucleic acid molecule comprised in the group consisting of
I) a nucleic acid molecule represented by SEQ ID NO: 1 and
II) a nucleic acid molecule having at least 100, preferably at least 143 for example 150, more preferably at least 195 for example 200, more preferably at least 250, even more preferably 355, most preferably 500 consecutive base pairs of a sequence described by SEQ ID NO: 1 wherein the at least 100 by comprise base pair 151 to 250 of SEQ ID NO: 1, the at least 143 by for example 150 comprise base pair 139 to 281 of SEQ ID NO: 1, the at least 195 by for example 200 by or 250 by comprise base pair 127 to 321 of SEQ ID NO: 1 and the at least 355 by for example 500 by comprise base pair 10 to 363 of SED ID NO: 1 respectively and
III) a nucleic acid molecule having an identity of at least 70%, preferably at least 80%, more preferably at least 85% or 90%, even more preferably at least 95%, 97%, 98% or 99%, most preferably 100% over a sequence of at least 100, preferably at least 143 for example 150, more preferably at least 195 for example 200, more preferably at least 250, more preferably 355, even more preferably 500, most preferably 793 consecutive base pairs of a sequence described by SEQ ID NO: 1 wherein the at least 100 by comprise base pair 151 to 250 of SEQ ID NO: 1, the at least 143 by for example 150 comprise base pair 139 to 281 of SEQ ID NO: 1, the at least 195 by for example 200 by or 250 by comprise base pair 127 to 321 of SEQ ID NO: 1 and the at least 355 by for example 500 by comprise base pair 10 to 363 of SED ID NO: 1 respectively and
IV) a nucleic acid molecule hybridizing under high stringent conditions with a nucleic acid molecule of at least 100, preferably 150, more preferably 200, even more preferably 250, most preferably 500 by hybridizing under stringent, preferably high stringent, more preferably very high stringent conditions with a nucleic acid molecule of at least 100, preferably at least 143 for example 150, more preferably at least 195 for example 200, more preferably at least 250, even more preferably 355, most preferably 500 consecutive base pairs of a sequence described by SEQ ID NO: 1 wherein the at least 100 by comprise base pair 151 to 250 of SEQ ID NO: 1, the at least 143 by for example 150 comprise base pair 139 to 281 of SEQ ID NO: 1, the at least 195 by for example 200 by or 250 by comprise base pair 127 to 321 of SEQ ID NO: 1 and the at least 355 by for example 500 by comprise base pair 10 to 363 of SED ID NO: 1 respectively and
V) a nucleic acid molecule able to form a secondary structure homologous, preferably identical to the secondary structure formed by SEQ ID NO: 1 and
VI) the complement of any of the sequences as defined in I) to V).

A skilled person is aware of various methods for functionally linking two or more nucleic acid molecules. Such methods may encompass restriction/ligation, ligase independent cloning, recombineering, recombination or synthesis. Other methods may be employed to functionally link two or more nucleic acid molecules.

The at least one regulatory nucleic acid molecule functional in a plant which is released from the recombinant microRNA precursor may be derived from a microRNA naturally occurring in a wild-type plant but being heterologous to the microRNA precursor as defined by SEQ ID NO: 1 or the derivatives thereof as defined above in II) to VI). The at least one regulatory nucleic acid molecule functional in a plant may also be derived from another small regulating RNA molecule naturally occurring in a wild-type plant such as a siRNA, to-siRNA or activating RNA and the like. The at least one regulatory nucleic acid molecule functional in a plant may also be an artificial sequence not naturally occurring in a plant and designed by man.

The recombinant microRNA precursor comprising the at least one regulatory nucleic acid molecule functional in a plant may be introduced into a plant cell, plant or part thereof as nucleic acid oligonucleotide, for example DNA or RNA which may or may not comprise derivatives of naturally occurring nucleic acids.

It may also be introduced into a plant comprised in an expression construct which may be comprised in a viral vector, a plasmid vector or binary vector. Preferred expression constructs and vectors are defined above.

The recombinant microRNA precursor molecule may be introduced into a plant cell, plant or part thereof by transient or stable transformation. The skilled person is well aware of numerous methods for transient or stable transformation of plant cells, plants or part thereof. Producing a plant as used herein comprises methods for stable transformation such as introducing a recombinant DNA construct into a plant or part thereof by means of Agrobacterium mediated transformation, protoplast transformation, particle bombardment or the like and optionally subsequent regeneration of a transgenic plant. It also comprises methods for transient transformation of a plant or part thereof such as viral infection or Agrobacterium infiltration. A skilled person is aware of further methods for stable and/or transient transformation of a plant or part thereof.

Approaches such as breeding methods or protoplast fusion might also be employed for production of a plant of the invention and are covered herewith. A preferred embodiment is the stable transformation, preferably stable Agrobacterium mediated transformation.

The at least one regulatory nucleic acid molecule functional in a plant which is released from the recombinant microRNA precursor of the invention upon processing of said precursor in the plant cell is binding to the target sequence, hence the regulatory nucleic acid molecule is at least partially complementary to the target sequence. The at least one regulatory nucleic acid molecule functional in a plant may be completely complementary to a target sequence and therefore not comprising any mismatch or it may comprise 1 or more, for example 1, or 2 or more, for example 2, or 3 or more, for example 3, or 4 or more, for example 4 mismatches when compared to the target sequence. In a preferred embodiment, the at least one regulatory nucleic acid molecule functional in a plant comprises no or 1 mismatch with the target sequence.

The sequence targeted by the at least one regulatory nucleic acid molecule functional in a plant may be comprised in the genomic DNA of the plant cell or in a RNA molecule. The target sequence may be comprised in a promoter sequence, a coding sequence, a non-coding RNA sequence, an intron, a 5′ or 3′ UTR or the like.

A further aspect of the invention at hand is the method as defined above wherein
a) the at least one regulatory nucleic acid molecule sequence heterologous to the microRNA precursor molecule is replacing by 225-245 or the respective basepairs in the nucleic acid molecule sequences as defined above in II) to VI) and
b) the at least one regulatory nucleic acid molecule star sequence complementary to the at least one regulatory nucleic acid molecule sequence is replacing by 159-179 of SEQ NO:1 or the respective basepairs in the nucleic acid molecule sequences as defined above in II) to VI) and
wherein a) and b) and the nucleic acid molecule separating a) and b) are able to from a stem loop structure.

A further embodiment of the invention is the method as defined above, wherein the sequence replacing by 225-245 or the respective basepairs in the nucleic acid molecule sequence as defined in above under point II) to VI) is selected from the group consisting of

a. a nucleic acid molecule represented by any of SEQ ID NO: 18, 19, 20 and/or 21 or a multitude thereof or

b. a nucleic acid molecule having at least 15 or 16, preferably 17, more preferably 18, even more preferably 19, most preferably 20 consecutive base pairs of a sequence described by any of SEQ ID NO: 18, 19, 20 and/or 21or a multitude thereof or

c. a nucleic acid molecule having an identity of at least 70%, preferably at least 75%, more preferably at least 80%, more preferably 90%, even more preferably 95% most preferably 97, 98 or 99% to the entire sequence of any of SEQ ID NO: 18, 19, 20 and/or 21 or a multitude thereof or

d. a nucleic acid molecule comprising 5 mismatches, preferably 4 mismatches, preferably 3 mismatches, more preferably 2 mismatches, most preferably one mismatch when compared to any of SEQ ID NO: 18, 19, 20 and/or 21or a multitude thereof,

e. a nucleic acid molecule hybridizing under high stringent conditions with a nucleic acid molecule described by any of SEQ ID NO: 18, 19, 20 and/or 21or a multitude thereof.

A further embodiment of the invention is the method as defined above wherein the at least one regulatory nucleic acid molecule sequence replacing by 225-245 and the at least one regulatory nucleic acid molecule star sequence complementary to the at least one regulatory nucleic acid molecule sequence consist of 20 or 21 or 22 by or a multiple of 20 or 21 or 22 by or a multiple thereof. In a preferred embodiment, the at least one regulatory nucleic acid molecule sequence consists of 21 by or a multiple thereof.

In one embodiment, the isolated nucleic acid molecule comprises one or more of the regulatory nucleic acid molecule sequences. In a preferred embodiment, the isolated nucleic acid molecule comprises between 1 and 50 of the regulatory nucleic acid molecule sequences. In a more preferred embodiment, the nucleic acid molecule comprises between 1 and 10, even more preferred between 1 and 5, in a most preferred embodiment it comprises 1 or 2, preferably 1 of the regulatory nucleic acid molecule sequences.

In a preferred embodiment, the method of the invention may be applied to a dicotyledonous plant. In an even more preferred embodiment of the method of invention, the method is applied to a plant of the family Fabacea, preferably the genus Glycine, most preferably the specie Glycine max.

The use of the isolated nucleic acid molecule as defined above or the expression construct as defined above for modulating expression of a target gene in a plant or part thereof is further comprised in the invention at hand.

A further embodiment of the invention is a process for the production of a transgenic plant comprising the steps of

1. providing an isolated nucleic acid as defined above, a plant expression construct and/or a plant expression vector as defined above and

2. introducing said isolated nucleic acid, plant expression construct and/or a plant expression vector into a plant cell or part of a plant and

3. regenerating a transgenic plant from said plant cell or part of a plant.

The invention further encompasses a method of conferring nematode resistance to a plant by making a transgenic plant capable of expressing a recombinant microRNA precursor comprising any of the sequences comprised in the group consisting of

a. a nucleic acid molecule represented by any of SEQ ID NO: 18, 19, 20 and/or 21 or a multitude thereof or

b. a nucleic acid molecule having at least 15 or 16, preferably 17, more preferably 18, even more preferably 19, most preferably 20 consecutive base pairs of a sequence described by any of SEQ ID NO: 18, 19, 20 and/or 21or a multitude thereof or

c. a nucleic acid molecule having an identity of at least 70%, preferably at least 75%, more preferably at least 80%, more preferably 90%, even more preferably 95% most preferably 97, 98 or 99% to the entire sequence of any of SEQ ID NO: 18, 19, 20 and/or 21 or a multitude thereof or

d. a nucleic acid molecule comprising 5 mismatches, preferably 4 mismatches, preferably 3 mismatches, more preferably 2 mismatches, most preferably one mismatch when compared to any of SEQ ID NO: 18, 19, 20 and/or 21or a multitude thereof,

e. a nucleic acid molecule hybridizing under high stringent conditions with a nucleic acid molecule described by any of SEQ ID NO: 18, 19, 20 and/or 21or a multitude thereof, said method comprising the steps of
(a) preparing an expression construct or expression vector comprising a nucleic acid encoding the recombinant microRNA precursor of the invention;
(b) transforming a recipient plant with said expression construct or expression vector;
(c) producing one or more transgenic offspring of said recipient plant; and
(d) selecting the offspring for resistance to nematode infection.

By applying the method of the invention of conferring nematode resistance to a plant, the transgenic plant regenerated is able to control nematode infection. The plant may show reduced nematode infection or complete resistance to nematode infection.

Producing a plant as used herein comprises methods for stable transformation such as introducing a recombinant DNA construct into a plant or part thereof by means of Agrobacterium mediated transformation, protoplast transformation, particle bombardment or the like and optionally subsequent regeneration of a transgenic plant. It also comprises methods for transient transformation of a plant or part thereof such as viral infection or Agrobacterium infiltration. A skilled person is aware of further methods for stable and/or transient transformation of a plant or part thereof. Approaches such as breeding methods or protoplast fusion might also be employed for production of a plant of the invention and are covered herewith. A preferred embodiment is the stable transformation, preferably Agrobacterium mediated transformation.

The process of the invention may be applied to any plant, for example gymnosperm or angiosperm, preferably angiosperm, for example dicotyledonous or monocotyledonous plants, preferably dicotyledonous plants. Preferred monocotyledonous plants are for example corn, wheat, rice, barley, sorghum, musa, sugarcane, miscanthus and brachypodium, especially preferred monocotyledonous plants are corn, wheat and rice. Preferred dicotyledonous plants are for example soy, rape seed, canola, linseed, cotton, potato, sugar beet, tagetes and Arabidopsis, especially preferred dicotyledonous plants are soy, rape seed, canola and potato. In a preferred embodiment, the process of the invention is applied to dicotyledonous plants, preferably of the family Fabacea, more preferably of the genus Glycine, most preferably the specie Glycine max.

A further embodiment of the invention is the isolated nucleic acid molecule, the plant expression construct or the plant expression vector as defined above, wherein the basepairs replacing the basepairs representing the microRNA molecule and microRNA star molecule as defined above are selected from

a. a nucleic acid molecule represented by any of SEQ ID NO: 18, 19, 20 and/or 21or a multitude thereof or

b. a nucleic acid molecule having at least 15 or 16, preferably 17, more preferably 18, even more preferably 19, most preferably 20 consecutive base pairs of a sequence described by any of SEQ ID NO: 18, 19, 20 and/or 21or a multitude thereof or

c. a nucleic acid molecule having an identity of at least 70%, preferably at least 75%, more preferably at least 80%, more preferably 90%, even more preferably 95% most preferably 97, 98 or 99% to the entire sequence of any of SEQ ID NO: 18, 19, 20 and/or 21or a multitude thereof or

d. a nucleic acid molecule comprising 5 mismatches, preferably 4 mismatches, preferably 3 mismatches, more preferably 2 mismatches, most preferably one mismatch when compared to any of SEQ ID NO: 18, 19, 20 and/or 21or a multitude thereof,

e. a nucleic acid molecule hybridizing under high stringent conditions with a nucleic acid molecule described by any of SEQ ID NO: 18, 19, 20 and/or 21or a multitude thereof.

As used herein, the term “control,” when used in the context of an infection, refers to the reduction or prevention of an infection. Reducing or preventing an infection by a nematode will cause a plant to have increased resistance to the nematode; however, such increased resistance does not imply that the plant necessarily has 100% resistance to infection. In preferred embodiments, the resistance to infection by a nematode in a resistant plant is greater than 10%, 20%, 30%, 40%, 50%, 60%, 70%, 80%, 90%, or 95% in comparison to a wild type plant that is not resistant to nematodes. Preferably the wild type plant is a plant of a similar, more preferably identical genotype as the plant having increased resistance to the nematode, but does not comprise a recombinant microRNA precursor directed to the target gene. The plant's resistance to infection by the nematode may be due to the death, sterility, arrest in development, or impaired mobility of the nematode upon exposure to the recombinant microRNA precursor specific to a plant gene having some effect on feeding site development, maintenance, or overall ability of the feeding site to provide nutrition to the nematode. The term “resistant to nematode infection” or “a plant having nematode resistance” as used herein refers to the ability of a plant, as compared to a wild type plant, to avoid infection by nematodes, to kill nematodes or to hamper, reduce or stop the development, growth or multiplication of nematodes. This might be achieved by an active process, e.g. by producing a substance detrimental to the nematode, or by a passive process, like having a reduced nutritional value for the nematode or not developing structures induced by the nematode feeding site like syncytia or giant cells. The level of nematode resistance of a plant can be determined in various ways, e.g. by counting the nematodes being able to establish parasitism on that plant, or measuring development times of nematodes, proportion of male and female nematodes or, for cyst nematodes, counting the number of cysts or nematode eggs produced on roots of an infected plant or plant assay system.

As used herein, the term “amount sufficient to inhibit expression” refers to a concentration or amount of the recombinant microRNA precursor that is sufficient to reduce levels or stability of mRNA or protein produced from a target gene in a plant. As used herein, “inhibiting expression” refers to the absence or observable decrease in the level of protein and/or mRNA product from a target gene. Inhibition of the plant target gene expression may result in lethality to the parasitic nematode, or such inhibition may delay or prevent entry into a particular developmental step (e.g., metamorphosis), if plant disease is associated with a particular stage of the parasitic nematode's life cycle. The consequences of inhibition can be confirmed by examination of the outward properties of the nematode (as presented below in the examples).

In accordance with the invention, a plant transcribes a recombinant microRNA precursor, which specifically inhibits expression of a plant target gene that effects nematode feeding site development, feeding site maintenance, nematode survival, nematode metamorphosis, or nematode reproduction. In a preferred embodiment, the recombinant microRNA precursor is encoded by an expression vector that has been transformed into an ancestor of the infected plant. More preferably, the expression vector comprises a nucleic acid encoding the recombinant microRNA precursor under the transcriptional control of a root specific promoter or a parasitic nematode induced feeding cell-specific promoter. Most preferably, the expression vector comprises a nucleic acid encoding the recombinant microRNA precursor under the transcriptional control of a parasitic nematode induced feeding cell-specific promoter.

Whether present in an extra-chromosomal non-replicating vector or a vector that is integrated into a chromosome, the polynucleotide preferably resides in a plant expression cassette. A plant expression cassette preferably contains regulatory sequences capable of driving gene expression in plant cells that are operatively linked so that each sequence can fulfill its function, for example, termination of transcription by polyadenylation signals. Preferred polyadenylation signals are those originating from Agrobacterium tumefaciens t-DNA such as the gene 3 known as octopine synthase of the Ti-plasmid pTiACH5 (Gielen et al., 1984, EMBO J. 3:835) or functional equivalents thereof, but also all other terminators functionally active in plants are suitable. As plant gene expression is very often not limited on transcriptional levels, a plant expression cassette preferably contains other operatively linked sequences like translational enhancers such as the overdrive-sequence containing the 5′-untranslated leader sequence from tobacco mosaic virus enhancing the polypeptide per RNA ratio (Gallie et al., 1987, Nucl. Acids Research 15:8693-8711). Examples of plant expression vectors include those detailed in: Becker, D. et al., 1992, New plant binary vectors with selectable markers located proximal to the left border, Plant Mol. Biol. 20:1195-1197; Bevan, M. W., 1984, Binary Agrobacterium vectors for plant transformation, Nucl. Acid. Res. 12:8711-8721; and Vectors for Gene Transfer in Higher Plants; in: Transgenic Plants, Vol. 1, Engineering and Utilization, eds.: Kung and R. Wu, Academic Press, 1993, S. 15-38.

Plant gene expression should be operatively linked to an appropriate promoter conferring gene expression in a temporal-preferred, spatial-preferred, cell type-preferred, and/or tissue-preferred manner. Promoters useful in the expression cassettes of the invention include any promoter that is capable of initiating transcription in a plant cell present in the plant's roots. Such promoters include, but are not limited to those that can be obtained from plants, plant viruses and bacteria that contain genes that are expressed in plants, such as Agrobacterium and Rhizobium. Preferably, the expression cassette of the invention comprises a root-specific promoter, a pathogen inducible promoter, or a nematode inducible promoter. More preferably the nematode inducible promoter is or a parasitic nematode feeding site-specific promoter. A parasitic nematode feeding site-specific promoter may be specific for syncytial cells or giant cells or specific for both kinds of cells. A promoter is inducible, if its activity, measured on the amount of RNA produced under control of the promoter, is at least 30%, 40%, 50% preferably at least 60%, 70%, 80%, 90% more preferred at least 100%, 200%, 300% higher in its induced state, than in its uninduced state. A promoter is cell-, tissue- or organ-specific, if its activity , measured on the amount of RNA produced under control of the promoter, is at least 30%, 40%, 50% preferably at least 60%, 70%, 80%, 90% more preferred at least 100%, 200%, 300% higher in a particular cell-type, tissue or organ, then in other cell-types or tissues of the same plant, preferably the other cell-types or tissues are cell types or tissues of the same plant organ, e.g. a root. In the case of organ specific promoters, the promoter activity has to be compared to the promoter activity in other plant organs, e.g. leaves, stems, flowers or seeds.

The promoter may be constitutive, inducible, developmental stage-preferred, cell type-preferred, tissue-preferred or organ-preferred. Constitutive promoters are active under most conditions. Non-limiting examples of constitutive promoters include the CaMV 19S and 35S promoters (Odell et al., 1985, Nature 313:810-812), the sX CaMV 35S promoter (Kay et al., 1987, Science 236:1299-1302), the Sep1 promoter, the rice actin promoter (McElroy et al., 1990, Plant Cell 2:163-171), the Arabidopsis actin promoter, the ubiquitin promoter (Christensen et al., 1989, Plant Molec. Biol. 18:675-689); pEmu (Last et al., 1991, Theor. Appl. Genet. 81:581-588), the figwort mosaic virus 35S promoter, the Smas promoter (Velten et al., 1984, EMBO J. 3:2723-2730), the GRP1-8 promoter, the cinnamyl alcohol dehydrogenase promoter (U.S. Pat. No. 5,683,439), promoters from the T-DNA of Agrobacterium, such as mannopine synthase, nopaline synthase, and octopine synthase, the small subunit of ribulose biphosphate carboxylase (ssuRUBISCO) promoter, and the like. Promoters that express the recombinant microRNA precursor in a cell that is contacted by parasitic nematodes are preferred. Alternatively, the promoter may drive expression of the recombinant microRNA precursor in a plant tissue remote from the site of contact with the nematode, and the recombinant microRNA precursor may then be transported by the plant to a cell that is contacted by the parasitic nematode, in particular cells of, or close by nematode feeding sites, e.g. syncytial cells or giant cells.

Inducible promoters are active under certain environmental conditions, such as the presence or absence of a nutrient or metabolite, heat or cold, light, pathogen attack, anaerobic conditions, and the like. For example, the promoters TobRB7, AtRPE, AtPyk10, Geminil9, and AtHMG1 have been shown to be induced by nematodes (for a review of nematode-inducible promoters, see Ann. Rev. Phytopathol. (2002) 40:191-219; see also U.S. Pat. No. 6,593,513). Method for isolating additional promoters, which are inducible by nematodes are set forth in U.S. Pat. Nos. 5,589,622 and 5,824,876. Other inducible promoters include the hsp80 promoter from Brassica, being inducible by heat shock; the PPDK promoter is induced by light; the PR-1 promoter from tobacco, Arabidopsis, and maize are inducible by infection with a pathogen; and the Adh1 promoter is induced by hypoxia and cold stress. Plant gene expression can also be facilitated via an inducible promoter (For review, see Gatz, 1997, Annu. Rev. Plant Physiol. Plant Mol. Biol. 48:89-108). Chemically inducible promoters are especially suitable if time-specific gene expression is desired. Non-limiting examples of such promoters are a salicylic acid inducible promoter

(PCT Application No. WO 95/19443), a tetracycline inducible promoter (Gatz et al., 1992, Plant J. 2:397-404) and an ethanol inducible promoter (PCT Application No. WO 93/21334).

Developmental stage-preferred promoters are preferentially expressed at certain stages of development. Tissue and organ preferred promoters include those that are preferentially expressed in certain tissues or organs, such as leaves, roots, seeds, or xylem. Examples of tissue preferred and organ preferred promoters include, but are not limited to fruit-preferred, ovule-preferred, male tissue-preferred, seed-preferred, integument-preferred, tuber-preferred, stalk-preferred, pericarp-preferred, and leaf-preferred, stigma-preferred, pollen-preferred, anther-preferred, a petal-preferred, sepal-preferred, pedicel-preferred, silique-preferred, stem-preferred, root-preferred promoters and the like. Seed preferred promoters are preferentially expressed during seed development and/or germination. For example, seed preferred promoters can be embryo-preferred, endosperm preferred and seed coat-preferred. See Thompson et al., 1989, BioEssays 10:108. Examples of seed preferred promoters include, but are not limited to cellulose synthase (celA), Cim1, gamma-zein, globulin-1, maize 19 kD zein (cZ19B1) and the like.

Other suitable tissue-preferred or organ-preferred promoters include, but are not limited to, the napin-gene promoter from rapeseed (U.S. Pat. No. 5,608,152), the USP-promoter from Vicia faba (Baeumlein et al., 1991, Mol Gen Genet. 225(3):459-67), the oleosin-promoter from Arabidopsis (PCT Application No. WO 98/45461), the phaseolin-promoter from Phaseolus vulgaris (U.S. Pat. No. 5,504,200), the Bce4-promoter from Brassica (PCT Application No. WO 91/13980), or the legumin B4 promoter (LeB4; Baeumlein et al., 1992, Plant Journal, 2(2):233-9), as well as promoters conferring seed specific expression in monocot plants like maize, barley, wheat, rye, rice, etc. Suitable promoters to note are the Ipt2 or Ipt1-gene promoter from barley (PCT Application No. WO 95/15389 and PCT Application No. WO 95/23230) or those described in PCT Application No. WO 99/16890 (promoters from the barley hordein-gene, rice glutelin gene, rice oryzin gene, rice prolamin gene, wheat gliadin gene, wheat glutelin gene, oat glutelin gene, Sorghum kasirin-gene, and rye secalin gene).

Other promoters useful in the expression cassettes of the invention include, but are not limited to, the major chlorophyll a/b binding protein promoter, histone promoters, the Ap3 promoter, the β-conglycin promoter, the napin promoter, the soybean lectin promoter, the maize 15 kD zein promoter, the 22 kD zein promoter, the 27 kD zein promoter, the g-zein promoter, the waxy, shrunken 1, shrunken 2, and bronze promoters, the Zm13 promoter (U.S. Pat. No. 5,086,169), the maize polygalacturonase promoters (PG) (U.S. Pat. Nos. 5,412,085 and 5,545,546), and the SGB6 promoter (U.S. Pat. No. 5,470,359), as well as synthetic or other natural promoters.

Of particular utility in the present invention are syncytia site preferred, or nematode feeding site induced, promoters, including, but not limited to promoters from the Mtn3-like promoter disclosed in WO 2008/095887, the Mtn21-like promoter disclosed in WO 2007/096275, the peroxidase-like promoter disclosed in WO 2008/077892, the trehalose-6-phosphate phosphatase-like promoter disclosed in WO 2008/071726 and the At5g12170-like promoter disclosed in WO 2008/095888.

Suitable methods for transforming or transfecting host cells including plant cells are well known in the art of plant biotechnology. Any method may be used to transform the recombinant expression vector into plant cells to yield the transgenic plants of the invention. General methods for transforming dicotyledenous plants are disclosed, for example, in U.S. Pat. Nos. 4,940,838; 5,464,763, and the like. Methods for transforming specific dicotyledenous plants, for example, cotton, are set forth in U.S. Pat. Nos. 5,004,863; 5,159,135; and 5,846,797. Soybean transformation methods are set forth in U.S. Pat. Nos. 4,992,375; 5,416,011; 5,569,834; 5,824,877; 6,384,301 and in EP 0301749B1 may be used. Transformation methods may include direct and indirect methods of transformation. Suitable direct methods include polyethylene glycol induced DNA uptake, liposome-mediated transformation (U.S. Pat. No. 4,536,475), biolistic methods using the gene gun (Fromm ME et al., Bio/Technology. 8(9):833-9, 1990; Gordon-Kamm et al. Plant Cell 2:603, 1990), electroporation, incubation of dry embryos in DNA-comprising solution, and micro-injection. In the case of these direct transformation methods, the plasmids used need not meet any particular requirements. Simple plasmids, such as those of the pUC series, pBR322, M13mp series, pACYC184 and the like can be used. If intact plants are to be regenerated from the transformed cells, an additional selectable marker gene is preferably located on the plasmid. The direct transformation techniques are equally suitable for dicotyledonous and monocotyledonous plants.

Transformation can also be carried out by bacterial infection by means of Agrobacterium (for example EP 0 116 718), viral infection by means of viral vectors (EP 0 067 553; U.S. Pat. No. 4,407,956; WO 95/34668; WO 93/03161) or by means of pollen (EP 0 270 356; WO 85/01856; U.S. Pat. No. 4,684,611). Agrobacterium based transformation techniques (especially for dicotyledonous plants) are well known in the art. The Agrobacterium strain (e.g., Agrobacterium tumefaciens or Agrobacterium rhizogenes) comprises a plasmid (Ti or Ri plasmid) and a T-DNA element which is transferred to the plant following infection with Agrobacterium. The T-DNA (transferred DNA) is integrated into the genome of the plant cell. The T-DNA may be localized on the Ri- or Ti-plasmid or is separately comprised in a so-called binary vector. Methods for the Agrobacterium-mediated transformation are described, for example, in Horsch RB et al. (1985) Science 225:1229. The Agrobacterium-mediated transformation is best suited to dicotyledonous plants but has also been adapted to monocotyledonous plants. The transformation of plants by Agrobacteria is described in, for example, White FF, Vectors for Gene Transfer in Higher Plants, Transgenic Plants, Vol. 1, Engineering and Utilization, edited by S. D. Kung and R. Wu, Academic Press, 1993, pp. 15 - 38; Jenes B et al. Techniques for Gene Transfer, Transgenic Plants, Vol. 1, Engineering and Utilization, edited by S. D. Kung and R. Wu, Academic Press, 1993, pp. 128-143; Potrykus (1991) Annu Rev Plant Physiol Plant Molec Biol 42:205-225. Transformation may result in transient or stable transformation and expression. Although a nucleotide sequence of the present invention can be inserted into any plant and plant cell falling within these broad classes, it is particularly useful in crop plant cells.

The transgenic plants of the invention may be crossed with similar transgenic plants or with transgenic plants lacking the nucleic acids of the invention or with non-transgenic plants, using known methods of plant breeding, to prepare seeds. Further, the transgenic plant of the present invention may comprise, and/or be crossed to another transgenic plant that comprises one or more nucleic acids, thus creating a “stack” of transgenes in the plant and/or its progeny. The seed is then planted to obtain a crossed fertile transgenic plant comprising the nucleic acid of the invention. The crossed fertile transgenic plant may have the particular expression cassette inherited through a female parent or through a male parent. The second plant may be an inbred plant. The crossed fertile transgenic may be a hybrid. Also included within the present invention are seeds of any of these crossed fertile transgenic plants. The seeds of this invention can be harvested from fertile transgenic plants and be used to grow progeny generations of transformed plants of this invention including hybrid plant lines comprising the DNA construct.

“Gene stacking” can also be accomplished by transferring two or more genes into the cell nucleus by plant transformation. Multiple genes may be introduced into the cell nucleus during transformation either sequentially or in unison. Multiple genes in plants or target pathogen species can be down-regulated by gene silencing mechanisms, by using a single transgene targeting multiple linked partial sequences of interest. Stacked, multiple genes under the control of individual promoters can also be over-expressed to attain a desired single or multiple pheno-types. Constructs containing gene stacks of both over-expressed genes and silenced targets can also be introduced into plants yielding single or multiple agronomically important phenotypes. In certain embodiments the nucleic acid sequences of the present invention can be stacked with any combination of polynucleotide sequences of interest to create desired pheno-types. The combinations can produce plants with a variety of trait combinations including but not limited to disease resistance, herbicide tolerance, yield enhancement, cold and drought tolerance. These stacked combinations can be created by any method including but not limited to cross breeding plants by conventional methods or by genetic transformation. If the traits are stacked by genetic transformation, the polynucleotide sequences of interest can be combined sequentially or simultaneously in any order. For example if two genes are to be introduced, the two sequences can be contained in separate transformation cassettes or on the same transformation cassette. The expression of the sequences can be driven by the same or different promoters.

Increased resistance to nematode infection is a general trait wished to be inherited into a wide variety of plants. The present invention may be used to reduce crop destruction by any plant parasitic nematode. Preferably, the parasitic nematodes belong to nematode families inducing giant or syncytial cells, such as Longidoridae, Trichodoridae, Heterodidae, Meloidogynidae, Pratylenchidae or Tylenchulidae. In particular in the families Heterodidae and Meloidogynidae.

When the parasitic nematodes are of the genus Globodera, exemplary targeted species include, without limitation, G. achilleae, G. artemisiae, G. hypolysi, G. mexicana, G. millefolii, G. mali, G. pallida, G. rostochiensis, G. tabacum, and G. virginiae. When the parasitic nematodes are of the genus Heterodera, exemplary targeted species include, without limitation, H. avenae, H. carotae, H. ciceri, H. cruciferae, H. delvii, H. elachista, H. filipjevi, H. gambiensis, H. glycines, H. goettingiana, H. graduni, H. humuli, H. hordecalis, H. latipons, H. major, H. medicaginis, H. oryzicola, H. pakistanensis, H. rosii, H. sacchari, H. schachtii, H. sorghi, H. trifolii, H. urticae, H. vigni and H. zeae. When the parasitic nematodes are of the genus Meloidogyne, exemplary targeted species include, without limitation, M. acronea, M. arabica, M. arenaria, M. artiellia, M. brevicauda, M. camelliae, M. chitwoodi, M. cofeicola, M. esigua, M. graminicola, M. hapla, M. incognita, M. indica, M. inornata, M. javanica, M. lini, M. mali, M. microcephala, M. microtyla, M. naasi, M. salasi and M. thamesi.

The following examples are not intended to limit the scope of the claims to the invention, but are rather intended to be exemplary of certain embodiments. Any variations in the exemplified methods that occur to the skilled artisan are intended to fall within the scope of the present invention.

### DEFINITIONS

Abbreviations: GFP—green fluorescence protein, GUS—beta-Glucuronidase, BAP—6-benzylaminopurine; 2,4-D-2,4-dichlorophenoxyacetic acid; MS—Murashige and Skoog medium; NAA—1-naphtaleneacetic acid; MES, 2-(N-morpholino-ethanesulfonic acid, IAA indole acetic acid; Kan: Kanamycin sulfate; GA3 —Gibberellic acid; Timentin™: ticarcillin disodium/clavulanate potassium.

It is to be understood that this invention is not limited to the particular methodology or protocols. It is also to be understood that the terminology used herein is for the purpose of describing particular embodiments only, and is not intended to limit the scope of the present invention which will be limited only by the appended claims. It must be noted that as used herein and in the appended claims, the singular forms “a,” “and,” and “the” include plural reference unless the context clearly dictates otherwise. Thus, for example, reference to “a vector” is a reference to one or more vectors and includes equivalents thereof known to those skilled in the art, and so forth. The term “about” is used herein to mean approximately, roughly, around, or in the region of.

When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). As used herein, the word “or” means any one member of a particular list and also includes any combination of members of that list. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof. For clarity, certain terms used in the specification are defined and used as follows:

Antiparallel: “Antiparallel” refers herein to two nucleotide sequences paired through hydrogen bonds between complementary base residues with phosphodiester bonds running in the 5′-3′ direction in one nucleotide sequence and in the 3′-5′ direction in the other nucleotide sequence.

Antisense: The term “antisense” refers to a nucleotide sequence that is inverted relative to its normal orientation for transcription or function and so expresses an RNA transcript that is complementary to a target gene mRNA molecule expressed within the host cell (e.g., it can hybridize to the target gene mRNA molecule or single stranded genomic DNA through Watson-Crick base pairing) or that is complementary to a target DNA molecule such as, for example genomic DNA present in the host cell.

Coding region: As used herein the term “coding region” when used in reference to a structural gene refers to the nucleotide sequences which encode the amino acids found in the nascent polypeptide as a result of translation of a mRNA molecule. The coding region is bounded, in eukaryotes, on the 5′-side by the nucleotide triplet “ATG” which encodes the initiator methionine and on the 3′-side by one of the three triplets which specify stop codons (i.e., TAA, TAG, TGA). In addition to containing introns, genomic forms of a gene may also include sequences located on both the 5′- and 3′-end of the sequences which are present on the RNA transcript. These sequences are referred to as “flanking” sequences or regions (these flanking sequences are located 5′ or 3′ to the non-translated sequences present on the mRNA transcript). The 5′-flanking region may contain regulatory sequences such as promoters and enhancers which control or influence the transcription of the gene. The 3′-flanking region may contain sequences which direct the termination of transcription, post-transcriptional cleavage and polyadenylation.

Complementary: “Complementary” or “complementarity” refers to two nucleotide sequences which comprise antiparallel nucleotide sequences capable of pairing with one another (by the base-pairing rules) upon formation of hydrogen bonds between the complementary base residues in the antiparallel nucleotide sequences. For example, the sequence 5′-AGT-3′ is complementary to the sequence 5′-ACT-3′. Complementarity can be “partial” or “total.” “Partial” complementarity is where one or more nucleic acid bases are not matched according to the base pairing rules. “Total” or “complete” complementarity between nucleic acid molecules is where each and every nucleic acid base is matched with another base under the base pairing rules.

The degree of complementarity between nucleic acid molecule strands has significant effects on the efficiency and strength of hybridization between nucleic acid molecule strands. A “complement” of a nucleic acid sequence as used herein refers to a nucleotide sequence whose nucleic acid molecules show total complementarity to the nucleic acid molecules of the nucleic acid sequence.

“Consecutive basepairs” as used herein means an uninterrupted sequence of nucleic acids. With reference to a fragment of a nucleic acid molecule, for example “50 consecutive base pairs of sequence 1” this would mean a sequence of 50 nucleic acids identical to sequence 1 not interrupted by a mismatch, a deletion or insertion.

Double-stranded RNA: A “double-stranded RNA” molecule or “dsRNA” molecule comprises a sense RNA fragment of a nucleotide sequence and an antisense RNA fragment of the nucleotide sequence, which both comprise nucleotide sequences complementary to one another, thereby allowing the sense and antisense RNA fragments to pair and form a double-stranded RNA molecule.

Endogenous: An “endogenous” nucleotide sequence refers to a nucleotide sequence, which is present in the genome of the untransformed plant cell.

Enhanced expression: “enhance” or “increase” the expression of a nucleic acid molecule in a plant cell are used equivalently herein and mean that the level of expression of the nucleic acid molecule in a plant, part of a plant or plant cell after applying a method of the present invention is higher than its expression in the plant, part of the plant or plant cell before applying the method, or compared to a reference plant lacking a recombinant nucleic acid molecule of the invention. For example, the reference plant is comprising the same construct which is only lacking the respective part complementary to at least a part of the precursor molecule comprising the srRNA sequence. The term “enhanced” or “increased” as used herein are synonymous and means herein higher, preferably significantly higher expression of the nucleic acid molecule to be expressed. As used herein, an “enhancement” or “increase” of the level of an agent such as a protein, mRNA or RNA means that the level is increased relative to a substantially identical plant, part of a plant or plant cell grown under substantially identical conditions, lacking a recombinant nucleic acid molecule of the invention, for example lacking the respective part complementary to at least a part of the precursor molecule comprising the srRNA sequence, the recombinant construct or recombinant vector of the invention. As used herein, “enhancement” or “increase” of the level of an agent, such as for example a preRNA, mRNA, rRNA, tRNA, snoRNA, snRNA expressed by the target gene and/or of the protein product encoded by it, means that the level is increased 50% or more, for example 100% or more, preferably 200% or more, more preferably 5 fold or more, even more preferably 10 fold or more, most preferably 20 fold or more for example 50 fold relative to a cell or organism lacking a recombinant nucleic acid molecule of the invention. The enhancement or increase can be determined by methods with which the skilled worker is familiar. Thus, the enhancement or increase of the nucleic acid or protein quantity can be determined for example by an immunological detection of the protein.

Moreover, techniques such as protein assay, fluorescence, Northern hybridization, nuclease protection assay, reverse transcription (quantitative RT-PCR), ELISA (enzyme-linked immunosorbent assay), Western blotting, radioimmunoassay (RIA) or other immunoassays and fluorescence-activated cell analysis (FACS) can be employed to measure a specific protein or RNA in a plant or plant cell. Depending on the type of the induced protein product, its activity or the effect on the phenotype of the organism or the cell may also be determined. Methods for determining the protein quantity are known to the skilled worker. Examples, which may be mentioned, are: the micro-Biuret method (Goa J (1953) Scand J Clin Lab Invest 5:218-222), the Folin-Ciocalteau method (Lowry OH et al. (1951) J Biol Chem 193:265-275) or measuring the absorption of CBB G-250 (Bradford MM (1976) Analyt Biochem 72:248-254). As one example for quantifying the activity of a protein, the detection of luciferase activity is described in the Examples below.

Expression: “Expression” may refer to the biosynthesis of a gene product, preferably to the transcription and/or translation of a nucleotide sequence, for example an endogenous gene or a heterologous gene, in a cell. For example, in the case of a structural gene, expression involves transcription of the structural gene into mRNA and—optionally—the subsequent translation of mRNA into one or more polypeptides. The term “expression” may also refer to the transcription of the DNA harboring an RNA molecule. Expression may further refer to the change of the steady state level of the respective RNA in a plant or part thereof for example due to change of the stability of the respective RNA.

Expression construct: “Expression construct” as used herein mean a DNA sequence capable of directing expression of a particular nucleotide sequence in an appropriate part of a plant or plant cell, comprising a promoter functional in said part of a plant or plant cell into which it will be introduced, operatively linked to the nucleotide sequence of interest which is—optionally—operatively linked to termination signals. If translation is required, it also typically comprises sequences required for proper translation of the nucleotide sequence. The coding region may code for a protein of interest but may also code for a functional RNA of interest, for example RNAa, siRNA, snoRNA, snRNA, microRNA, to-siRNA or any other noncoding regulatory RNA, in the sense or antisense direction. The expression construct comprising the nucleotide sequence of interest may be chimeric, meaning that one or more of its components is heterologous with respect to one or more of its other components. The expression construct may also be one, which is naturally occurring but has been obtained in a recombinant form useful for heterologous expression. Typically, however, the expression construct is heterologous with respect to the host, i.e., the particular DNA sequence of the expression construct does not occur naturally in the host cell and must have been introduced into the host cell or an ancestor of the host cell by a transformation event. The expression of the nucleotide sequence in the expression construct may be under the control of a constitutive promoter or of an inducible promoter, which initiates transcription only when the host cell is exposed to some particular external stimulus. In the case of a plant, the promoter can also be specific to a particular tissue or organ or stage of development.

Foreign: The term “foreign” refers to any nucleic acid molecule (e.g., gene sequence) which is introduced into the genome of a cell by experimental manipulations and may include sequences found in that cell so long as the introduced sequence contains some modification (e.g., a point mutation, the presence of a selectable marker gene, etc.) and is therefore distinct relative to the naturally-occurring sequence.

Functional linkage: The term “functional linkage” or “functionally linked” is to be understood as meaning, for example, the sequential arrangement of a regulatory element (e.g. a promoter) with a nucleic acid sequence to be expressed and, if appropriate, further regulatory elements (such as e.g., a terminator or an enhancer) in such a way that each of the regulatory elements can fulfill its intended function to allow, modify, facilitate or otherwise influence expression of said nucleic acid sequence. As a synonym the wording “operable linkage” or “operably linked” may be used. The expression may result depending on the arrangement of the nucleic acid sequences in relation to sense or antisense RNA. To this end, direct linkage in the chemical sense is not necessarily required. Genetic control sequences such as, for example, enhancer sequences, can also exert their function on the target sequence from positions which are further away, or indeed from other DNA molecules. Preferred arrangements are those in which the nucleic acid sequence to be expressed recombinantly is positioned behind the sequence acting as promoter, so that the two sequences are linked covalently to each other. The distance between the promoter sequence and the nucleic acid sequence to be expressed recombinantly is preferably less than 200 base pairs, especially preferably less than 100 base pairs, very especially preferably less than 50 base pairs. In a preferred embodiment, the nucleic acid sequence to be transcribed is located behind the promoter in such a way that the transcription start is identical with the desired beginning of the chimeric RNA of the invention. Functional linkage, and an expression construct, can be generated by means of customary recombination and cloning techniques as described (e.g., in Maniatis T, Fritsch E F and Sambrook J (1989) Molecular Cloning: A Laboratory Manual, 2nd Ed., Cold Spring Harbor Laboratory, Cold Spring Harbor (N.Y.); Silhavy et al. (1984) Experiments with Gene Fusions, Cold Spring Harbor Laboratory, Cold Spring Harbor (N.Y.); Ausubel et al. (1987) Current Protocols in Molecular Biology, Greene Publishing Assoc. and Wiley Interscience; Gelvin et al. (Eds) (1990) Plant Molecular Biology Manual; Kluwer Academic Publisher, Dordrecht, The Netherlands). However, further sequences, which, for example, act as a linker with specific cleavage sites for restriction enzymes, or as a signal peptide, may also be positioned between the two sequences. The insertion of sequences may also lead to the expression of fusion proteins. Preferably, the expression construct, consisting of a linkage of a regulatory region for example a promoter and nucleic acid sequence to be expressed, can exist in a vector-integrated form and be inserted into a plant genome, for example by transformation.

Gene: The term “gene” refers to a region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (downstream) the coding region (open reading frame, ORF) as well as, where applicable, intervening sequences (i.e., introns) between individual coding regions (i.e., exons). The term “structural gene” as used herein is intended to mean a DNA sequence that is transcribed into mRNA which is then translated into a sequence of amino acids characteristic of a specific polypeptide.

Genome and genomic DNA: The terms “genome” or “genomic DNA” is referring to the heritable genetic information of a host organism. Said genomic DNA comprises the DNA of the nucleus (also referred to as chromosomal DNA) but also the DNA of the plastids (e.g., chloroplasts) and other cellular organelles (e.g., mitochondria). Preferably the terms genome or genomic DNA is referring to the chromosomal DNA of the nucleus.

Heterologous: The term “heterologous” with respect to a nucleic acid molecule or DNA refers to a nucleic acid molecule which is operably linked to, or is manipulated to become operably linked to, a second nucleic acid molecule to which it is not operably linked in nature, or to which it is operably linked at a different location in nature. A heterologous expression construct comprising a nucleic acid molecule and one or more regulatory nucleic acid molecule (such as a promoter or a transcription termination signal) linked thereto for example is a constructs originating by experimental manipulations in which either a) said nucleic acid molecule, or b) said regulatory nucleic acid molecule or c) both (i.e. (a) and (b)) is not located in its natural (native) genetic environment or has been modified by experimental manipulations, an example of a modification being a substitution, addition, deletion, inversion or insertion of one or more nucleotide residues.

Natural genetic environment refers to the natural chromosomal locus in the organism of origin, or to the presence in a genomic library. In the case of a genomic library, the natural genetic environment of the sequence of the nucleic acid molecule is preferably retained, at least in part.

The environment flanks the nucleic acid sequence at least at one side and has a sequence of at least 50 bp, preferably at least 500 bp, especially preferably at least 1,000 bp, very especially preferably at least 5,000 bp, in length. A naturally occurring expression construct—for example the naturally occurring combination of a promoter with the corresponding gene—becomes a transgenic expression construct when it is modified by non-natural, synthetic “artificial” methods such as, for example, mutagenization. Such methods have been described (U.S. Pat. No. 5,565,350; WO 00/15815). For example a protein encoding nucleic acid molecule operably linked to a promoter, which is not the native promoter of this molecule, is considered to be heterologous with respect to the promoter. Preferably, heterologous DNA is not endogenous to or not naturally associated with the cell into which it is introduced, but has been obtained from another cell or has been synthesized. Heterologous DNA also includes an endogenous DNA sequence, which contains some modification, non-naturally occurring, multiple copies of an endogenous DNA sequence, or a DNA sequence which is not naturally associated with another DNA sequence physically linked thereto. Generally, although not necessarily, heterologous DNA encodes RNA or proteins that are not normally produced by the cell into which it is expressed.

Hybridization: The term “hybridization” as used herein includes “any process by which a strand of nucleic acid molecule joins with a complementary strand through base pairing.” (J. Coombs (1994) Dictionary of Biotechnology, Stockton Press, New York). Hybridization and the strength of hybridization (i.e., the strength of the association between the nucleic acid molecules) is impacted by such factors as the degree of complementarity between the nucleic acid molecules, stringency of the conditions involved, the Tm of the formed hybrid, and the G:C ratio within the nucleic acid molecules. As used herein, the term “Tm” is used in reference to the “melting temperature.” The melting temperature is the temperature at which a population of double-stranded nucleic acid molecules becomes half dissociated into single strands. The equation for calculating the Tm of nucleic acid molecules is well known in the art. As indicated by standard references, a simple estimate of the Tm value may be calculated by the equation: Tm=81.5+0.41(% G+C), when a nucleic acid molecule is in aqueous solution at 1 M NaCl [see e.g., Anderson and Young, Quantitative Filter Hybridization, in Nucleic Acid Hybridization (1985)]. Other references include more sophisticated computations, which take structural as well as sequence characteristics into account for the calculation of Tm. Stringent conditions, are known to those skilled in the art and can be found in Current Protocols in Molecular Biology, John Wiley & Sons, N.Y. (1989), 6.3.1-6.3.6.

Medium stringent conditions when used in reference to nucleic acid hybridization comprise conditions equivalent to binding or hybridization at 68° C. in a solution consisting of 5× SSPE (43.8 g/L NaCl, 6.9 g/L NaH2PO4.H2O and 1.85 g/L EDTA, pH adjusted to 7.4 with NaOH), 1% SDS, 5× Denhardt's reagent [50× Denhardt's contains the following per 500 mL 5 g Ficoll (Type 400, Pharmacia), 5 g BSA (Fraction V; Sigma)] and 100 μg/mL denatured salmon sperm DNA followed by washing (preferably for one times 15 minutes, more preferably two times 15 minutes, more preferably three time 15 minutes) in a solution comprising 1×SSC (1×SSC is 0.15 M NaCl plus 0.015 M sodium citrate) and 0.1% SDS at room temperature or—preferably 37° C.—when a DNA probe of preferably about 100 to about 500 nucleotides in length is employed.

High stringent conditions when used in reference to nucleic acid hybridization comprise conditions equivalent to binding or hybridization at 68° C. in a solution consisting of 5× SSPE (43.8 g/L NaCl, 6.9 g/L NaH2PO4.H20 and 1.85 g/L EDTA, pH adjusted to 7.4 with NaOH), 1% SDS, 5× Denhardt's reagent [50× Denhardt's contains the following per 500 mL 5 g Ficoll (Type 400, Pharmacia), 5 g BSA (Fraction V; Sigma)] and 100 pg/mL denatured salmon sperm DNA followed by washing (preferably for one times 15 minutes, more preferably two times 15 minutes, more preferably three time 15 minutes) in a solution comprising 0.1×SSC (1×SSC is 0.15 M NaCl plus 0.015 M sodium citrate) and 1% SDS at room temperature or—preferably 37° C.—when a DNA probe of preferably about 100 to about 500 nucleotides in length is employed. Very high stringent conditions when used in reference to nucleic acid hybridization comprise conditions equivalent to binding or hybridization at 68° C. in a solution consisting of 5× SSPE, 1% SDS, 5× Denhardt's reagent and 100 μg/mL denatured salmon sperm DNA followed by washing (preferably for one times 15 minutes, more preferably two times 15 minutes, more preferably three time 15 minutes) in a solution comprising 0.1× SSC, and 1% SDS at 68° C., when a probe of preferably about 100 to about 500 nucleotides in length is employed.

“Identity”: “Identity” when used in respect to the comparison of two or more nucleic acid or amino acid molecules means that the sequences of said molecules share a certain degree of sequence similarity, the sequences being partially identical.

To determine the percentage identity (homology is herein used interchangeably) of two amino acid sequences or of two nucleic acid molecules, the sequences are written one underneath the other for an optimal comparison (for example gaps may be inserted into the sequence of a protein or of a nucleic acid in order to generate an optimal alignment with the other protein or the other nucleic acid).

The amino acid residues or nucleic acid molecules at the corresponding amino acid positions or nucleotide positions are then compared. If a position in one sequence is occupied by the same amino acid residue or the same nucleic acid molecule as the corresponding position in the other sequence, the molecules are homologous at this position (i.e. amino acid or nucleic acid “homology” as used in the present context corresponds to amino acid or nucleic acid “identity”. The percentage identity between the two sequences is a function of the number of identical positions shared by the sequences (i.e. % homology=number of identical positions/total number of positions×100). The terms “homology” and “identity” are thus to be considered as synonyms.

For the determination of the percentage identity of two or more amino acids or of two or more nucleotide sequences several computer software programs have been developed. The identity of two or more sequences can be calculated with for example the software fasta, which presently has been used in the version fasta 3 (W. R. Pearson and D. J. Lipman, PNAS 85, 2444(1988); W. R. Pearson, Methods in Enzymology 183, 63 (1990); W. R. Pearson and D. J. Lipman, PNAS 85, 2444 (1988); W. R. Pearson, Enzymology 183, 63 (1990)). Another useful program for the calculation of identities of different sequences is the standard blast program, which is included in the Biomax pedant software (Biomax, Munich, Federal Republic of Germany). This leads unfortunately sometimes to suboptimal results since blast does not always include complete sequences of the subject and the query. Nevertheless as this program is very efficient it can be used for the comparison of a huge number of sequences. The following settings are typically used for such a comparisons of sequences:

-p Program Name [String]; -d Database [String]; default=nr; -i Query File [File In]; default=stdin; -e Expectation value (E) [Real]; default=10.0; -m alignment view options: 0=pairwise; 1=query-anchored showing identities; 2=query-anchored no identities; 3=flat query-anchored, show identities; 4=flat query-anchored, no identities; 5=query-anchored no identities and blunt ends; 6=flat query-anchored, no identities and blunt ends; 7=XML Blast output; 8=tabular; 9 tabular with comment lines [Integer]; default=0; -o BLAST report Output File [File Out] Optional; default=stdout; -F Filter query sequence (DUST with blastn, SEG with others) [String]; default=T; -G Cost to open a gap (zero invokes default behavior) [Integer]; default=0; -E Cost to extend a gap (zero invokes default behavior) [Integer]; default=0; -X X dropoff value for gapped alignment (in bits) (zero invokes default behavior); blastn 30, megablast 20, tblastx 0, all others 15 [Integer]; default=0; -I Show GI's in deflines [T/F]; default =F; -q Penalty for a nucleotide mismatch (blastn only) [Integer]; default=-3; -r Reward for a nucleotide match (blastn only) [Integer]; default=1; -v Number of database sequences to show one-line descriptions for (V) [Integer]; default=500; -b Number of database sequence to show alignments for (B) [Integer]; default=250; -f Threshold for extending hits, default if zero; blastp 11, blastn 0, blastx 12, tblastn 13; tblastx 13, megablast 0 [Integer]; default=0; -g Perfom gapped alignment (not available with tblastx) [T/F]; default=T; -Q Query Genetic code to use [Integer]; default=1; -D DB Genetic code (for tblast[nx] only) [Integer]; default=1; -a Number of processors to use [Integer]; default=1; -O SeqAlign file [File Out] Optional; -J Believe the query defline [T/F]; default=F; -M Matrix [String]; default=BLOSUM62; -W Word size, default if zero (blastn 11, megablast 28, all others 3) [Integer]; default=0; -z Effective length of the database (use zero for the real size) [Real]; default=0; -K Number of best hits from a region to keep (off by default, if used a value of 100 is recommended) [Integer]; default=0; -P 0 for multiple hit, 1 for single hit [Integer]; default=0; -Y Effective length of the search space (use zero for the real size) [Real]; default=0; -S Query strands to search against database (for blast[nx], and tblastx); 3 is both, 1 is top, 2 is bottom [Integer]; default=3; -T Produce HTML output [T/F]; default=F; -I Restrict search of database to list of GI's [String] Optional; -U Use lower case filtering of FASTA sequence [T/F] Optional; default=F; -y X dropoff value for ungapped extensions in bits (0.0 invokes default behavior); blastn 20, megablast 10, all others 7 [Real]; default=0.0; -Z X dropoff value for final gapped alignment in bits (0.0 invokes default behavior); blastn/megablast 50, tblastx 0, all others 25 [Integer]; default=0; -R PSI-TBLASTN checkpoint file [File In] Optional; -n MegaBlast search [T/F]; default=F; -L Location on query sequence [String] Optional; -A Multiple Hits window size, default if zero (blastn/megablast 0, all others 40

[Integer]; default=0; -w Frame shift penalty (00F algorithm for blastx) [Integer]; default =0; -t Length of the largest intron allowed in tblastn for linking HSPs (0 disables linking) [Integer]; default=0.

Results of high quality are reached by using the algorithm of Needleman and Wunsch or Smith and Waterman. Therefore programs based on said algorithms are preferred. Advantageously the comparisons of sequences can be done with the program PileUp (J. Mol. Evolution., 25, 351 (1987), Higgins et al., CABIOS 5, 151 (1989)) or preferably with the programs “Gap” and “Needle”, which are both based on the algorithms of Needleman and Wunsch (J. Mol. Biol. 48; 443 (1970)), and “BestFit”, which is based on the algorithm of Smith and Waterman (Adv. Appl. Math. 2; 482 (1981)). “Gap” and “BestFit” are part of the GCG software-package (Genetics Computer Group, 575 Science Drive, Madison, Wis., USA 53711 (1991); Altschul et al., (Nucleic Acids Res. 25, 3389 (1997)), “Needle” is part of the The European Molecular Biology Open Software Suite (EMBOSS) (Trends in Genetics 16 (6), 276 (2000)). Therefore preferably the calculations to determine the percentages of sequence identity are done with the programs

“Gap” or “Needle” over the whole range of the sequences. The following standard adjustments for the comparison of nucleic acid sequences were used for “Needle”: matrix: EDNAFULL, Gap_penalty: 10.0, Extend_penalty: 0.5. The following standard adjustments for the comparison of nucleic acid sequences were used for “Gap”: gap weight: 50, length weight: 3, average match: 10.000, average mismatch: 0.000.

For example a sequence, which is said to have 80% identity with sequence SEQ ID NO: 1 at the nucleic acid level is understood as meaning a sequence which, upon comparison with the sequence represented by SEQ ID NO: 1 by the above program “Needle” with the above parameter set, has a 80% identity. Preferably the identity is calculated on the complete length of the query sequence, for example SEQ ID NO: 1 or base pair 139 to 281 of SEQ ID NO: 1.

Isogenic: organisms (e.g., plants), which are genetically identical, except that they may differ by the presence or absence of a heterologous DNA sequence.

Isolated: The term “isolated” as used herein means that a material has been removed by the hand of man and exists apart from its original, native environment and is therefore not a product of nature. An isolated material or molecule (such as a DNA molecule or enzyme) may exist in a purified form or may exist in a non-native environment such as, for example, in a transgenic host cell. For example, a naturally occurring polynucleotide or polypeptide present in a living plant is not isolated, but the same polynucleotide or polypeptide, separated from some or all of the coexisting materials in the natural system, is isolated. Such polynucleotides can be part of a vector and/or such polynucleotides or polypeptides could be part of a composition, and would be isolated in that such a vector or composition is not part of its original environment. Preferably, the term “isolated” when used in relation to a nucleic acid molecule, as in “an isolated nucleic acid sequence” refers to a nucleic acid sequence that is identified and separated from at least one contaminant nucleic acid molecule with which it is ordinarily associated in its natural source. Isolated nucleic acid molecule is nucleic acid molecule present in a form or setting that is different from that in which it is found in nature. In contrast, non-isolated nucleic acid molecules are nucleic acid molecules such as DNA and RNA, which are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs, which encode a multitude of proteins. However, an isolated nucleic acid sequence comprising for example SEQ ID NO: 1 includes, by way of example, such nucleic acid sequences in cells which ordinarily contain SEQ ID NO:1 where the nucleic acid sequence is in a chromosomal or extrachromosomal location different from that of natural cells, or is otherwise flanked by a different nucleic acid sequence than that found in nature. The isolated nucleic acid sequence may be present in single-stranded or double-stranded form. When an isolated nucleic acid sequence is to be utilized to express a protein, the nucleic acid sequence will contain at a minimum at least a portion of the sense or coding strand (i.e., the nucleic acid sequence may be single-stranded). Alternatively, it may contain both the sense and anti-sense strands (i.e., the nucleic acid sequence may be double-stranded).

Minimal Promoter: promoter elements, particularly a TATA element, that are inactive or that have greatly reduced promoter activity in the absence of upstream activation. In the presence of a suitable transcription factor, the minimal promoter functions to permit transcription.

“Modulating the expression of a gene” in a plant means the increase, enhancement, repression or downregulation of the expression of a target gene compared to a wild-type or reference plant to which the method of the invention has not been applied.

Non-coding: The term “non-coding” refers to sequences of nucleic acid molecules that do not encode part or all of an expressed protein. Non-coding sequences include but are not limited to introns, enhancers, promoter regions, 3′ untranslated regions, and 5′ untranslated regions.

Nucleic acids and nucleotides: The terms “Nucleic Acids” and “Nucleotides” refer to naturally occurring or synthetic or artificial nucleic acid or nucleotides. The terms “nucleic acids” and “nucleotides” comprise deoxyribonucleotides or ribonucleotides or any nucleotide analogue and polymers or hybrids thereof in either single- or double-stranded, sense or antisense form. Unless otherwise indicated, a particular nucleic acid sequence also implicitly encompasses conservatively modified variants thereof (e.g., degenerate codon substitutions) and complementary sequences, as well as the sequence explicitly indicated. The term “nucleic acid” is used inter-changeably herein with “gene”, “cDNA”, “mRNA”, “oligonucleotide,” and “polynucleotide”. Nucleotide analogues include nucleotides having modifications in the chemical structure of the base, sugar and/or phosphate, including, but not limited to, 5-position pyrimidine modifications, 8-position purine modifications, modifications at cytosine exocyclic amines, substitution of 5-bromo-uracil, and the like; and 2′-position sugar modifications, including but not limited to, sugar-modified ribonucleotides in which the 2′—OH is replaced by a group selected from H, OR, R, halo, SH, SR, NH2, NHR, NR2, or CN. Short hairpin RNAs (shRNAs) also can comprise non-natural elements such as non-natural bases, e.g., ionosin and xanthine, non-natural sugars, e.g., 2′-methoxy ribose, or non-natural phosphodiester linkages, e.g., methylphosphonates, phosphorothioates and peptides.

Nucleic acid sequence: The phrase “nucleic acid sequence” refers to a single or double-stranded polymer of deoxyribonucleotide or ribonucleotide bases read from the 5′- to the 3′-end. It includes chromosomal DNA, self-replicating plasmids, infectious polymers of DNA or RNA and DNA or RNA that performs a primarily structural role. “Nucleic acid sequence” also refers to a consecutive list of abbreviations, letters, characters or words, which represent nucleotides. In one embodiment, a nucleic acid can be a “probe” which is a relatively short nucleic acid, usually less than 100 nucleotides in length. Often a nucleic acid probe is from about 50 nucleotides in length to about 10 nucleotides in length. A “target region” of a nucleic acid is a portion of a nucleic acid that is identified to be of interest. A “coding region” of a nucleic acid is the portion of the nucleic acid, which is transcribed and translated in a sequence-specific manner to produce into a particular polypeptide or protein when placed under the control of appropriate regulatory sequences. The coding region is said to encode such a polypeptide or protein.

Oligonucleotide: The term “oligonucleotide” refers to an oligomer or polymer of ribonucleic acid (RNA) or deoxyribonucleic acid (DNA) or mimetics thereof, as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases. An oligonucleotide preferably includes two or more nucleomonomers covalently coupled to each other by linkages (e.g., phosphodiesters) or substitute linkages.

Overhang: An “overhang” is a relatively short single-stranded nucleotide sequence on the 5′- or 3′-hydroxyl end of a double-stranded oligonucleotide molecule (also referred to as an “extension,” “protruding end,” or “sticky end”).

Plant: is generally understood as meaning any eukaryotic single-or multi-celled organism or a cell, tissue, organ, part or propagation material (such as seeds or fruit) of same which is capable of photosynthesis. Included for the purpose of the invention are all genera and species of higher and lower plants of the Plant Kingdom. Annual, perennial, monocotyledonous and dicotyledonous plants are preferred. The term includes the mature plants, seed, shoots and seedlings and their derived parts, propagation material (such as seeds or microspores), plant organs, tissue, protoplasts, callus and other cultures, for example cell cultures, and any other type of plant cell grouping to give functional or structural units. Mature plants refer to plants at any desired developmental stage beyond that of the seedling. Seedling refers to a young immature plant at an early developmental stage. Annual, biennial, monocotyledonous and dicotyledonous plants are preferred host organisms for the generation of transgenic plants. The expression of genes is furthermore advantageous in all ornamental plants, useful or ornamental trees, flowers, cut flowers, shrubs or lawns. Plants which may be mentioned by way of example but not by limitation are angiosperms, bryophytes such as, for example, Hepaticae (liverworts) and Musci (mosses); Pteridophytes such as ferns, horsetail and club mosses; gymnosperms such as conifers, cycads, ginkgo and Gnetatae; algae such as Chlorophyceae, Phaeophpyceae, Rhodophyceae, Myxophyceae, Xanthophyceae, Bacillariophyceae (diatoms), and Euglenophyceae. Preferred are plants which are used for food or feed purpose such as the families of the Leguminosae such as pea, alfalfa and soya; Gramineae such as rice, maize, wheat, barley, sorghum, millet, rye, triticale, or oats; the family of the Umbelliferae, especially the genus Daucus, very especially the species carota (carrot) and Apium, very especially the species Graveolens dulce (celery) and many others; the family of the Solanaceae, especially the genus Lycopersicon, very especially the species esculentum (tomato) and the genus Solanum, very especially the species tuberosum (potato) and melongena (egg plant), and many others (such as tobacco); and the genus Capsicum, very especially the species annuum (peppers) and many others; the family of the Leguminosae, especially the genus Glycine, very especially the species max (soybean), alfalfa, pea, lucerne, beans or peanut and many others; and the family of the Cruciferae (Brassicacae), especially the genus Brassica, very especially the species napus (oil seed rape), campestris (beet), oleracea cv Tastie (cabbage), oleracea cv Snowball Y (cauliflower) and oleracea cv Emperor (broccoli); and of the genus Arabidopsis, very especially the species thaliana and many others; the family of the Compositae, especially the genus Lactuca, very especially the species sativa (lettuce) and many others; the family of the Asteraceae such as sunflower, Tagetes, lettuce or Calendula and many other; the family of the Cucurbitaceae such as melon, pumpkin/squash or zucchini, and linseed. Further preferred are cotton, sugar cane, hemp, flax, chillies, and the various tree, nut and wine species.

Polypeptide: The terms “polypeptide”, “peptide”, “oligopeptide”, “polypeptide”, “gene product”, “expression product” and “protein” are used interchangeably herein to refer to a polymer or oligomer of consecutive amino acid residues.

Pre-protein: Protein, which is normally targeted to a cellular organelle, such as a chloroplast, and still comprising its transit peptide.

Primary transcript: The term “primary transcript” as used herein refers to a premature RNA transcript of a gene. A “primary transcript” for example still comprises introns and/or is not yet comprising a polyA tail or a cap structure and/or is missing other modifications necessary for its correct function as transcript such as for example trimming or editing.

Promoter: The terms “promoter”, or “promoter sequence” are equivalents and as used herein, refer to a DNA sequence which when ligated to a nucleotide sequence of interest is capable of controlling the transcription of the nucleotide sequence of interest into RNA. Such promoters can for example be found in the following public databases http://www.grassius.org/grasspromdb.html, http://mendel.cs.rhul.ac.uk/mendel.php?topic=plantprom, http://ppdb.gene.nagoya-u.ac.jp/cgi-bin/index.cgi. Promoters listed there may be addressed with the methods of the invention and are herewith included by reference. A promoter is located 5′ (i.e., upstream), proximal to the transcriptional start site of a nucleotide sequence of interest whose transcription into mRNA it controls, and provides a site for specific binding by RNA polymerase and other transcription factors for initiation of transcription. Said promoter comprises for example the at least 10 kb, for example 5 kb or 2 kb proximal to the transcription start site. It may also comprise the at least 1500 by proximal to the transcriptional start site, preferably the at least 1000 bp, more preferably the at least 500 bp, even more preferably the at least 400 bp, the at least 300 bp, the at least 200 by or the at least 100 bp. In a further preferred embodiment, the promoter comprises the at least 50 by proximal to the transcription start site, for example, at least 25 bp. The promoter does not comprise exon and/or intron regions or 5′ untranslated regions. The promoter may for example be heterologous or homologous to the respective plant. A polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is not naturally associated with the promoter (e.g. a genetically engineered coding sequence or an allele from a different ecotype or variety). Suitable promoters can be derived from genes of the host cells where expression should occur or from pathogens for this host cells (e.g., plants or plant pathogens like plant viruses). A plant specific promoter is a promoter suitable for regulating expression in a plant. It may be derived from a plant but also from plant pathogens or it might be a synthetic promoter designed by man. If a promoter is an inducible promoter, then the rate of transcription increases in response to an inducing agent. Also, the promoter may be regulated in a tissue-specific or tissue preferred manner such that it is only or predominantly active in transcribing the associated coding region in a specific tissue type(s) such as leaves, roots or meristem. The term “tissue specific” as it applies to a promoter refers to a promoter that is capable of directing selective expression of a nucleotide sequence of interest to a specific type of tissue (e.g., petals) in the relative absence of expression of the same nucleotide sequence of interest in a different type of tissue (e.g., roots). Tissue specificity of a promoter may be evaluated by, for example, operably linking a reporter gene to the promoter sequence to generate a reporter construct, introducing the reporter construct into the genome of a plant such that the reporter construct is integrated into every tissue of the resulting transgenic plant, and detecting the expression of the reporter gene (e.g., detecting mRNA, protein, or the activity of a protein encoded by the reporter gene) in different tissues of the transgenic plant. The detection of a greater level of expression of the reporter gene in one or more tissues relative to the level of expression of the reporter gene in other tissues shows that the promoter is specific for the tissues in which greater levels of expression are detected. The term “cell type specific” as applied to a promoter refers to a promoter, which is capable of directing selective expression of a nucleotide sequence of interest in a specific type of cell in the relative absence of expression of the same nucleotide sequence of interest in a different type of cell within the same tissue. The term “cell type specific” when applied to a promoter also means a promoter capable of promoting selective expression of a nucleotide sequence of interest in a region within a single tissue. Cell type specificity of a promoter may be assessed using methods well known in the art, e.g., GUS activity staining, GFP protein or immunohistochemical staining. The term “constitutive” when made in reference to a promoter or the expression derived from a promoter means that the promoter is capable of directing transcription of an operably linked nucleic acid molecule in the absence of a stimulus (e.g., heat shock, chemicals, light, etc.) in the majority of plant tissues and cells throughout substantially the entire lifespan of a plant or part of a plant. Typically, constitutive promoters are capable of directing expression of a transgene in substantially any cell and any tissue.

Promoter specificity: The term “specificity” when referring to a promoter means the pattern of expression conferred by the respective promoter. The specificity describes the tissues and/or developmental status of a plant or part thereof, in which the promoter is conferring expression of the nucleic acid molecule under the control of the respective promoter. Specificity of a promoter may also comprise the environmental conditions, under which the promoter may be activated or down-regulated such as induction or repression by biological or environmental stresses such as cold, drought, wounding or infection.

Purified: As used herein, the term “purified” refers to molecules, either nucleic or amino acid sequences that are removed from their natural environment, isolated or separated. “Substantially purified” molecules are at least 60% free, preferably at least 75% free, and more preferably at least 90% free from other components with which they are naturally associated. A purified nucleic acid sequence may be an isolated nucleic acid sequence.

Recombinant: The term “recombinant” with respect to nucleic acid molecules refers to nucleic acid molecules produced by recombinant DNA techniques. Recombinant nucleic acid molecules may also comprise molecules, which as such does not exist in nature but are modified, changed, mutated or otherwise manipulated by man. Preferably, a “recombinant nucleic acid molecule” is a non-naturally occurring nucleic acid molecule that differs in sequence from a naturally occurring nucleic acid molecule by at least one nucleic acid. A “recombinant nucleic acid molecule” may also comprise a “recombinant construct” which comprises, preferably operably linked, a sequence of nucleic acid molecules not naturally occurring in that order. Preferred methods for producing said recombinant nucleic acid molecule may comprise cloning techniques, directed or non-directed mutagenesis, synthesis or recombination techniques.

“Recombinant microRNA precursor molecule” is to be understood as a microRNA precursor molecule which has been modified by replacing the microRNA molecule, hence replacing the microRNA and the microRNA star sequence, naturally comprised in said precursor molecule, therefore being homologous to the precursor molecule by at least one other microRNA molecule or regulatory nucleic acid molecule, hence at least one other microRNA and microRNA star or regulatory nucleic acid or regulatory nucleic acid star sequence heterologous to the microRNA precursor. A microRNA molecule or regulatory nucleic acid molecule heterologous to the microRNA precursor molecule means a microRNA molecule or regulatory nucleic acid molecule which is not naturally linked to the microRNA precursor molecule, hence which is not occurring in a wild type plant. The heterologous microRNA molecule or regulatory nucleic acid molecule may be a microRNA molecule or regulatory nucleic acid molecule naturally occurring in a wild-type plant but in said wild-type plant being comprised in another microRNA precursor molecule or the heterologous microRNA molecule or regulatory nucleic acid molecule may be man made and not occurring in a wild-type plant. The heterologous microRNA molecule or regulatory nucleic acid molecule may target a target gene which is targeted by other microRNAs in a wild-type plant or may target a target gene which is not targeted by microRNA molecules or regulatory nucleic acid molecules in a wild-type plant. The heterologous microRNA molecule or regulatory nucleic acid molecule may target the coding region of a target gene, an intron or 5″or 3′UTR of a target gene or a regulatory sequences such as a promoter of a target gene.

“Regulatory nucleic acid molecule” as used herein means preferably small regulating RNA molecules or srRNA molecules and is understood as molecules consisting of nucleic acids or derivatives thereof. They may be double-stranded or single-stranded and are between about 15 and about 30 bp, for example between 15 and 30 bp, more preferred between about 19 and about 26 bp, for example between 19 and 26 bp, even more preferred between about 20 and about 25 by for example between 20 and 25 bp. In an especially preferred embodiment the oligonucleotides are between about 21 and about 24 bp, for example between 21 and 24 bp. In a most preferred embodiment, the small nucleic acid molecules are about 21 by and about 24 bp, for example 21 by and 24 bp. The regulatory nucleic acid molecules modulate steady state levels of RNAs, for example mRNAs or other regulatory nucleic acid molecules in a plant cell, a plant or part thereof by modulating transcription or stability of the respective RNA targeted by the regulatory nucleic acid molecule.

“Repress” or “downregulate” the expression of a nucleic acid molecule in a plant cell are used equivalently herein and mean that the level of expression of the nucleic acid molecule in a plant, part of a plant or plant cell after applying a method of the present invention is lower than its expression in the plant, part of the plant or plant cell before applying the method, or compared to a reference plant lacking a recombinant nucleic acid molecule of the invention. For example, the reference plant comprises the same construct which is only lacking the respective precursor molecule. The term “repressed” or “downregulated” as used herein are synonymous and means herein lower, preferably significantly lower expression of the nucleic acid molecule to be expressed. As used herein, a “repression” or “downregulation” of the level of an agent such as a protein, mRNA or RNA means that the level is reduced relative to a substantially identical plant, part of a plant or plant cell grown under substantially identical conditions, lacking a recombinant nucleic acid molecule of the invention, for example lacking the region complementary to at least a part of the precursor molecule of the srRNA, the recombinant construct or recombinant vector of the invention. As used herein, “repression” or “downregulation” of the level of an agent, such as for example a preRNA, mRNA, rRNA, tRNA, snoRNA, snRNA expressed by the target gene and/or of the protein product encoded by it, means that the amount is reduced 10% or more, for example 20% or more, preferably 30% or more, more preferably 50% or more, even more preferably 70% or more, most preferably 80% or more for example 90% relative to a cell or organism lacking a recombinant nucleic acid molecule of the invention. The repression or downregulation can be determined by methods with which the skilled worker is familiar. Thus, the enhancement or increase of the nucleic acid or protein quantity can be determined for example by an immunological detection of the protein. Moreover, techniques such as protein assay, fluorescence, Northern hybridization, nuclease protection assay, reverse transcription (quantitative RT-PCR), ELISA (enzyme-linked immunosorbent assay), Western blotting, radioimmunoassay (RIA) or other immunoassays and fluorescence-activated cell analysis (FACS) can be employed to measure a specific protein or RNA in a plant or plant cell. Depending on the type of the induced protein product, its activity or the effect on the phenotype of the organism or the cell may also be determined. Methods for determining the protein quantity are known to the skilled worker. Examples, which may be mentioned, are: the micro-Biuret method (Goa J (1953) Scand J Clin Lab Invest 5:218-222), the Folin-Ciocalteau method (Lowry OH et al. (1951) J Biol Chem 193:265-275) or measuring the absorption of CBB G-250 (Bradford MM (1976) Analyt Biochem 72:248-254). As one example for quantifying the activity of a protein, the detection of luciferase activity is described in the Examples below.

Sense: The term “sense” is understood to mean a nucleic acid molecule having a sequence which is complementary or identical to a target sequence, for example a sequence which binds to a protein transcription factor and which is involved in the expression of a given gene. According to a preferred embodiment, the nucleic acid molecule comprises a gene of interest and elements allowing the expression of the said gene of interest.

The secondary structure of a nucleic acid molecule refers to the basepairing interactions within a single molecule or set of interacting molecules, and can be represented as a list of bases which are paired in a nucleic acid molecule. Single stranded RNA forms complicated base-pairing interactions due to its increased ability to form hydrogen bonds stemming from the extra hydroxyl group in the ribose sugar. Secondary structures comprise for example stem-loop-structure also known as hairpin structures. This structure occurs when two regions of the same strand, partially or completely complementary in nucleotide sequence when read in opposite directions, base-pair to form a double helix that ends in an unpaired loop.

Tools for the prediction of the secondary structure of nucleic acid molecules are for example CentroidFold (Michiaki Hamada, Hisanori Kiryu, Kengo Sato, Toutai Mituyama, Kiyoshi Asai (2009). “Predictions of RNA secondary structure using generalized centroid estimators”. Bioinformatics 25 (4): 465-473), CONTRAfold (Do C B, Woods D A, Batzoglou S (2006). “CONTRA-fold: RNA secondary structure prediction without physics-based models”. Bioinformatics 22 (14): e90-8), KineFold (Xayaphoummine A, Bucher T, Isambert H (2005). “Kinefold web server for RNA/DNA folding path and structure prediction including pseudoknots and knots”. Nucleic Acids Res. 33 (Web Server issue): W605-10), Mfold (Zuker M, Stiegler P (1981). “Optimal computer folding of large RNA sequences using thermodynamics and auxiliary information”. Nucleic Acids Res. 9 (1): 133-48.), Pknots (Rivas E, Eddy S R (1999). “A dynamic programming algorithm for RNA structure prediction including pseudoknots”. J. Mol. Biol. 285 (5): 2053-68), PknotsRG (Reeder J, Steffen P, Giegerich R (2007). “pknotsRG: RNA pseudoknot folding including near-optimal structures and sliding windows”. Nucleic Acids Res. 35 (Web Server issue): W320-4), RNAfold (I. L. Hofacker, W. Fontana, P. F. Stadler, S. Bonhoeffer, M. Tacker, P. Schuster (1994). “Fast Folding and Comparison of RNA Secondary Structures.”. Monatshefte f. Chemie 125: 167-188), RNAshapes (R. Giegerich, B.Voβ, M. Rehmsmeier (2004). “Abstract shapes of RNA.”. Nucleic Acids Res. 32 (16): 4843-4851), RNAstructure (D. H. Mathews, M. D. Disney, J. L. Childs, S. J. Schroeder, M. Zuker, D. H. Turner (2004). “Incorporating chemical modification constraints into a dynamic programming algorothm for prediction of RNA secondary structure.” Proceedings of the National Academy of Sciences, USA 101: 7287-7292.), Sfold (Ding Y, Chan C Y, Lawrence C E (2004). “Sfold web server for statistical folding and rational design of nucleic acids”. Nucleic Acids Res. 32 (Web Server issue): W135-41) or UNAFold (Markham N R, Zuker M (2008). “UNAFold: software for nucleic acid folding and hybridization.”. Methods Mol Biol 453: 3-31).

Significant increase or decrease: An increase or decrease, for example in enzymatic activity or in gene expression, that is larger than the margin of error inherent in the measurement technique, preferably an increase or decrease by about 2-fold or greater of the activity of the control enzyme or expression in the control cell, more preferably an increase or decrease by about 5-fold or greater, and most preferably an increase or decrease by about 10-fold or greater.

Substantially complementary: In its broadest sense, the term “substantially complementary”, when used herein with respect to a nucleotide sequence in relation to a reference or target nucleotide sequence, means a nucleotide sequence having a percentage of identity between the substantially complementary nucleotide sequence and the exact complementary sequence of said reference or target nucleotide sequence of at least 60%, more desirably at least 70%, more desirably at least 80% or 85%, preferably at least 90%, more preferably at least 93%, still more preferably at least 95% or 96%, yet still more preferably at least 97% or 98%, yet still more preferably at least 99% or most preferably 100% (the later being equivalent to the term “identical” in this context). Preferably identity is assessed over a length of at least 19 nucleotides, preferably at least 50 nucleotides, more preferably the entire length of the nucleic acid sequence to said reference sequence (if not specified otherwise below). Sequence comparisons are carried out using default GAP analysis with the University of Wisconsin GCG, SEQWEB application of GAP, based on the algorithm of Needleman and Wunsch (Needleman and Wunsch (1970) J Mol. Biol. 48: 443-453; as defined above). A nucleotide sequence “substantially complementary ” to a reference nucleotide sequence hybridizes to the reference nucleotide sequence under medium stringent conditions, preferably high stringent conditions, most preferably very high stringent conditions (as defined above).

Transgene: The term “transgene” as used herein refers to any nucleic acid sequence, which is introduced into the genome of a cell by experimental manipulations. A transgene may be an “endogenous DNA sequence,” or a “heterologous DNA sequence” (i.e., “foreign DNA”). The term “endogenous DNA sequence” refers to a nucleotide sequence, which is naturally found in the cell into which it is introduced so long as it does not contain some modification (e.g., a point mutation, the presence of a selectable marker gene, etc.) relative to the naturally-occurring sequence.

Transgenic: The term transgenic when referring to an organism means transformed, preferably stably transformed, with a recombinant DNA molecule that preferably comprises a suitable promoter operatively linked to a DNA sequence of interest.

Vector: As used herein, the term “vector” refers to a nucleic acid molecule capable of transporting another nucleic acid molecule to which it has been linked. One type of vector is a genomic integrated vector, or “integrated vector”, which can become integrated into the chromosomal DNA of the host cell. Another type of vector is an episomal vector, i.e., a nucleic acid molecule capable of extra-chromosomal replication. Vectors capable of directing the expression of genes to which they are operatively linked are referred to herein as “expression vectors”. In the present specification, “plasmid” and “vector” are used interchangeably unless otherwise clear from the context. Expression vectors designed to produce RNAs as described herein in vitro or in vivo may contain sequences recognized by any RNA polymerase, including mitochondrial RNA polymerase, RNA pol I, RNA pol II, and RNA pol III. These vectors can be used to transcribe the desired RNA molecule in the cell according to this invention. A plant transformation vector is to be understood as a vector suitable in the process of plant transformation.

Wild-type: The term “wild-type”, “natural” or “natural origin” means with respect to an organism, polypeptide, or nucleic acid sequence, that said organism is naturally occurring or available in at least one naturally occurring organism which is not changed, mutated, or otherwise manipulated by man.

### EXAMPLES

### Chemicals and Common Methods

Unless indicated otherwise, cloning procedures carried out for the purposes of the present invention including restriction digest, agarose gel electrophoresis, purification of nucleic acids, Ligation of nucleic acids, transformation, selection and cultivation of bacterial cells were performed as described (Sambrook et al., 1989). Sequence analyses of recombinant DNA were performed with a laser fluorescence DNA sequencer (Applied Biosystems, Foster City, Calif., USA) using the Sanger technology (Sanger et al., 1977). Unless described otherwise, chemicals and reagents were obtained from Sigma Aldrich (Sigma Aldrich, St. Louis, USA), from Promega (Madison, Wis., USA), Duchefa (Haarlem, The Netherlands) or Invitrogen (Carlsbad, Calif., USA). Restriction endonucleases were from New England Biolabs (Ipswich, Mass., USA) or Roche Diagnostics GmbH (Penzberg, Germany). Oligonucleotides were synthesized by Eurofins MWG Operon (Ebersberg, Germany).

### Example 1

### Identification of Soybean miR166 Precursor (Gm Pre-miR166)

Gm pre-miR166h (SEQ ID NO: 1): Hyseq 47124994
gatttcgtctctcaaactcgtttgtgctgagagaaccaagggtttcttcccttgcagagaagaaaatttggatacatatggttaagtttatt
tagatatcttgtttgttctttcatctttctcagatatggtttggaaatgggagattggggatgatgggaatgttgtttggctcgagaaaaag
ctttaaaggttggattttgaggctatccctttatgtgatctcggaccaggcttcattcccgtcaaccttatctctctctcttgagatcttct
cccatggaggggtgatggcttatgtatataggtttcccagctgtagcatctttagggtttgagattcctaaccatctttacttcctgtcaag
tttcagtccatgtggttggcttcattttttcagcccaggtgaatagagaaaagtgctgagaagatgcatgaagttaagatgaattaatggat
ctaaaacataacaaaattggtggcacacaatgcatacagaattttcctttcatttgttggttatcacttcatcgtttttcttgcttgtttct
accaaaataaaaggcgaaagagggagtgttgtggtggtggttgggctagcctgaaaaattgtaattcctaccaaggttttaaaaaatatcag
taacaggaatttcaatcacaatatcaagctttgtgggcgattgcatcttgtggtgacatcatcggtcatatttttccacaatgtcaggaatc
acaaccacactttaaaacctttatacaattttcctaaacaaaaaaaaaaaaaaaaaa

Note:
miR166 is from nucleotide 225-245, and miR166* is from nucleotide 159-179.
Gm-miR166 sequence: 5′ ucggaccaggcuucauucccc 3′ (SEQ ID NO: 2), and the last nucleotide is different from gm_miR166h.

This EST clone was identified by using soybean miR166 nucleotide sequence (5′ ucggaccaggcuucauucccc 3′) BLAST against BPS proprietary soybean Hyseq database Hyseq_Soybean_EST.nt. Hyseq clone 47124994 was retrieved with an E value 4e-04. Sequence analysis indicates that it contains both miR166 sequence (20/21 identical to gm_miR166) and miR166* sequence (19/21 identical to gm_miR166 and folds into a typical miRNA stem-loop structure with miR166 and miR166* regions as part of the stem. Therefore we predict that Hyseq 47124994 is an EST clone for Gm_miR166 precursor. We named it Gm pre-miR166h. The 352 by fragment from nucleotide 10-361in Hyseq 47124994 was used as the precursor for expressing Gm-miR166 or artificial Gm-miR166 in plants.

The genomic fragment corresponding to the 352bp Gm pre-miR166h was obtained by PCR using genomic DNA from soybean W82.

Genomic pre-miR166
(SED ID NO: 3)
Tctcaaactcgtttgtgctgagagaaccaagggtttcttcccttgcagagaagaaaatttggatacatatggttaagtttatttagatatctt
gtttgttctttcatctttctcagatatggtttggaaatgggagattggggatgatgggaatgttgtttggctcgaggtaactgcatggtcttaatt
ttgttcatcttttgaagctttaatttatttatgggtttcaatcttttttgatcccttgaaacagaaaaagctttaaaggttggattttgaggctatccc
tttatgtgatctcggaccaggcttcattcccgtcaaccttatctctctctcttgagatcttctcccatggaggggtgatggcttatggtaattaat
taaggcgatgtcaaaacaagactcttgtgatagatatatctggtcaatgatgacattaaaactctcttgtttgttattttttatcggtaaatatgt
tacttgttaaaatattagttttgttattaagattgatctgtaattttttttcttaaccatccaatctattatatatctcctatacttatttgttttgttt
gtgtttgtaaattcagacattgcatgggacaaaacttgaaacatttgtcaagaactctattgaacaagatgcctttgaccttgttgtaattggtgcttt
gttttttgtcatctttcttatggctccattatactttatatatcttcatttctttcaacatctctctcgatcgtgagttaatttaacggttgagacttga
gatttgtgctattatatattatgtctatctaaaaggatcaggctccaaaatctttatatagtatataattatttttcttggattatgattagtgagtaat
tggtattaacttttttaagttaaatattctgcgttgagaaacccaaggatctttgaaattctgtattttgtcagtatataggtttcccagctgtagc
atctttagggtttgagattcctaaccatctttacttcctgtc

By comparing to its cDNA sequence in Hyseq 47124994, we identified two introns located within this 1018 by genomic sequence. Nucleotide 2-169 are the exon 1, followed by 170-253 as intron 1, 254-375 as exon 2, and 376-953 as intron 2. Nucleotide 954-1018 is partial Exon 3. Interestingly miR166* is separated into two parts by intron1. The first part contains 20 by from 150-169, and the second part contains the last nucleotide in miR166* located at position 254. MiR166 is located in Exon 2 from position 300-320.

Gm pre-miR166h is a new miR166 precursor in soybean but share certain homology with other soybean miR166 precursors. Among all the known soybean miR166 precursors gm pre-miR166h shares the highest homology (68%) with the sequence #1087 published in patent US2005144669_A1 by Reinhart, Brenda and David Bartel, et al. 119 out of the 174 nucleotides in sequence #1087 are identical to gm prw-miR166h precursor we identified here.

### Example 2

### Proof-of-Concept of Making Artificial miRNA using Gm Pre-miR166 Targeting Phytoene Desaturase (PDS)

The native plant microRNA precursor has been engineered according to (Schwab et al., (2006), to produce artificial miRNA which specifically down-regulated target gene expression. A further method for the design of functional microRNAs is the tool WMD3 as described by Stephan Ossowski et al (2008). The tool is available in the internet with the following URL http://wmd3.weigelworld.org/cgi-bin/webapp.cgi).

We used the cDNA gm_miR166h precursor to express artificial microRNA miRPDS3 in soybean driven by parsley ubiquitin promoter. The 21-nt miR166 sequence was replaced with a 21-nt sequence miRPDS3 5′ tcatatgtgttcttcagttat 3′ (SEQ ID NO: 4) targeting to a conserved region in plant PDS genes. The gm_miR166h miR166* region was replaced by miRPDS3* sequence 5′ aactgacgaacacatatgaga 3′ (SEQ ID NO: 5). A control vector with wild-type gm_miR166h sequence was used as control in this experiment. Both plant expression vectors MW120 (miR166h) and MW121 (miRPDS3) were transformed into Agrobacterium and used in soybean TRAP roots transformation. Transgenic TRAP roots were collected and the PDS expression level was detected using Tagman assay on AB17900. Twelve independent transgenic events from both MW120 and MW121 were analyzed. PDS Tagman assay result showed that the PDS transcripts in soybean TRAP roots was reduced 70% in miRPDS3 construct MW121 compared to the control construct MW120, with the average of PDS transcripts level of 2.90 in MW121 events and 9.83 in MW121 events. The difference of PDS transcripts level is statistically significant with the P value of 0.03.

Construct MW120 and MW121 were also used to express anti-PDS artificial miRNA miRPDS3 in Arabidopsis and soybean whole plants transformation. In Arabidopsis, 12% of the transgenic plants (16 out of 138 plants) transformed with MW121 displayed a severe photo-bleaching phenotype, typical for PDS deficiency in plants. As a control, all the plants transformed with MW120 remained green. In soybean, bleaching phenotype was also observed occasionally but the frequency and degree of phenotype is not consistent.

### Example 3

### Characterization of Different Length of Gm Pre-miR166

Two shortened versions of the soy miR166h precursor were designed and tested. A 143 by and a 195 by version were designed. Both versions were designed to express the let-7 microRNA.

The 143 by version of Gm pri-miR166h consists of bases 130 to 272 of the original 355 by Gm pri-miR166h sequence (SEQ ID NO: 10). The final 143 by soy miR166h precursor sequence (143 by Gm pri-miR166h) is as follows with the artificial let-7 microRNA, 5′ TGAGGTAGTAGGTTGTATAGT3′ (SEQ ID NO: 6) sequence underlined.

ATGGGAGATTGGGGATGAACTATACATCCTACTACCACAGAAAAAGCTTTAAAGGTTGGAT
TTTGAGGCTATCCCTTTATGTGATCTGAGGTAGTAGGTTGTATAGTTCAACCTTATCTCTCT
CTCTTGAGATCTTCTCCCAT

The 195 by version of Gm pri-miR166h consists of bases 118 to 312 of the original 355 by Gm pri-miR166h sequence (SEQ ID NO: 10). The final 195 by soy miR166h precursor sequence (143 by Gm pri-miR166h) is as follows with the artificial let-7 microRNA sequence underlined.

TATGGTTTGGAAATGGGAGATTGGGGATGAACTATACATCCTACTACCACAGAAAAAGCTT
TAAAGGTTGGATTTTGAGGCTATCCCTTTATGTGATCTGAGGTAGTAGGTTGTATAGTTCAA
CCTTATCTCTCTCTCTTGAGATCTTCTCCCATGGAGGGGTGATGGCTTATGTATATAGGTTT
CCCAGCTGTA

Total RNA was extracted from the transgenic roots. The chimeric RNA/DNA 3′ adapter “R5” with sequence P-AUGCGGTGGTGGCTGAGCGGGCTGGCAAGGC-idT where the first 4 bases are RNA and the remainder DNA with an inverted thymidine at the 3′ end was ligated to total RNA using T4 RNA polymerase. This 3′ ligated RNA was then used as template for one step RT-PCR using the forward oligo “let-7 -3” with the sequence 5′ TGAGGTAGTAGGTTGTAT 3′ which is identical to the predicted miRNA sequence absent bases corresponding to the 3 nucleotides on the 3′ end of the predicted miRNA, and the reverse oligo “JMprim357” with sequence GCCTTGCCAGCCCGCTCAG that is complementary to the 3′ adapter. RT-PCR was performed using the kit SuperScript III One-Step RT-PCR System with Plantinum Taq DNA Polymerase (Cat. No. 12574-026) from Invitrogen, with the following conditions: 1 cycle, 30 minutes at 55° C.; 1 cycle, 2 minutes at 94° C.; 40 cycles, 15 seconds at 94° C., 30 seconds at 50° C., 20 seconds at 68° C.; and 5 minutes at 68° C. . A portion of the RT-PCR product was run on a 15% acrylamide/TBE gel alongside the Low Molecular Weight DNA Ladder (Cat. No. N3234S) from New England Biolabs to determine presence and size of the PCR amplified product.

Both shortened versions of Gm pri-miR166h produced a single band of the expected size indicating that they are functional and producing the correct microRNA.

### Example 4

### Use of Gm Pre-miR166 to Make miRNA Stacking Constructs

Two soy miR166h precursors were stacked in tandem that produce one transcript that when processed produces two different mature microRNAs. The stacked microRNA cassettes were made with the two 355 by and two 195bp versions of the soy miR166h precursor.

The sequence of the stacked 355 by precursors is as follows (SEQ ID NO: 7). Each precursor is expressing a different miRNA. The first precursor is expressing miR let-7 and the second precursor is expressing an artificial miRNA targeting the nematode gene let-70, 5′ TGGATGGTCAGAAAGAAGACGT3′ (SEQ ID NO: 8). The two precursors are separated by a Swal restriction site. The miRNA sequence in each precursor is underlined.

TCTCAAACTCGTTTGTGCTGAGAGAACCAAGGGTTTCTTCCCTTGCAGAGAAGAAAATTTG
GATACATATGGTTAAGTTTATTTAGATATCTTGTTTGTTCTTTCATCTTTCTCAGATATGGTTT
GGAAATGGGAGATTGGGGATGAACTATACATCCTACTACCACAGAAAAAGCTTTAAAGGTT
GGATTTTGAGGCTATCCCTTTATGTGATCTGAGGTAGTAGGTTGTATAGTTCAACCTTATCT
CTCTCTCTTGAGATCTTCTCCCATGGAGGGGTGATGGCTTATGTATATAGGTTTCCCAGCT
GTAGCATCTTTAGGGTTTGAGATTCCTAACCATCTTTACTTCCTGTATTTAAATTCTCAAACT
CGTTTGTGCTGAGAGAACCAAGGGTTTCTTCCCTTGCAGAGAAGAAAATTTGGATACATAT
GGTTAAGTTTATTTAGATATCTTGTTTGTTCTTTCATCTTTCTCAGATATGGTTTGGAAATGG
GAGATTGGGGATGACGTCTTCTATCTGACCATACAGAAAAAGCTTTAAAGGTTGGATTTTGA
GGCTATCCCTTTATGTGATCTGGATGGTCAGAAAGAAGACGTCAACCTTATCTCTCTCTCTT
GAGATCTTCTCCCATGGAGGGGTGATGGCTTATGTATATAGGTTTCCCAGCTGTAGCATCT
TTAGGGTTTGAGATTCCTAACCATCTTTACTTCCTGT

The sequence of the stacked 195 by precursors is as follows (SEQ ID NO: 9). Each precursor is expressing a different miRNA. The first precursor is expressing miR let-7 and the second precursor is expressing an artificial miRNA targeting the nematode gene let-7. The two precursors are separated by a Swal restriction site. The miRNA sequence in each precursor is underlined.

TATGGTTTGGAAATGGGAGATTGGGGATGAACTATACATCCTACTACCACAGAAAAAGCTT
TAAAGGTTGGATTTTGAGGCTATCCCTTTATGTGATCTGAGGTAGTAGGTTGTATAGTTCAA
CCTTATCTCTCTCTCTTGAGATCTTCTCCCATGGAGGGGTGATGGCTTATGTATATAGGTTT
CCCAGCTGTAATTTAAATTATGGTTTGGAAATGGGAGATTGGGGATGAACTCTTCTATCTGA
CCATACAGAAAAAGCTTTAAAGGTTGGATTTTGAGGCTATCCCTTTATGTGATCTGGATGGT
CAGAAAGAAGACGTCAACCTTATCTCTCTCTCTTGAGATCTTCTCCCATGGAGGGGTGATG
GCTTATGTATATAGGTTTCCCAGCTGTA

Transgenic roots were produced for both stacked miRNA cassettes. Total RNA was extracted from soybean roots expressing these constructs and used for small RNA sequencing with Illumina technology. The results demonstrated both let-7 and let-70 artificial miRNA are expressed as expected and the let-7 miRNA is expressed at higher level (˜30 fold) than the let-70 miRNA.

original 355 bp Gm pri-miR166h sequence
(SEQ ID NO: 10)
TCTCAAACTCGTTTGTGCTGAGAGAACCAAGGGTTTCTTCCCTTGCAGAGAAGAAAATTTG
GATACATATGGTTAAGTTTATTTAGATATCTTGTTTGTTCTTTCATCTTTCTCAGATATGGTTT
GGAAATGGGAGATTGGGGATGATGGGAATGTTGTTTGGCTCGAGAAAAAGCTTTAAAGGTT
GGATTTTGAGGCTATCCCTTTATGTGATCTCGGACCAGGCTTCATTCCCGTCAACCTTATCT
CTCTCTCTTGAGATCTTCTCCCATGGAGGGGTGATGGCTTATGTATATAGGTTTCCCAGCT
GTAGCATCTTTAGGGTTTGAGATTCCTAACCATCTTTACTTCCTGT

### Example 5

### Engineered miRNAs for Nematode Control—Vector Construction

DNA synthesis using an external DNA synthesis company was used to isolate DNA fragments used to construct the binary vectors described in Table 1 and discussed in Example 2. The described DNA synthesis products were cloned into DNA synthesis company proprietary vectors and inserts were confirmed by sequencing. The synthesized miRNA precursors described by the sequences pr-GM pre-miR166h CDPK-152 (SEQ ID NO: 11), pr-GM pre-miR166h Hg inx-3-937 (SEQ ID NO: 12) and pr-2x pre-miR opr3-541; ZF-40 (SEQ ID NO:13) were isolated using this method. When expressed in the plant the miRNA precursor sequence described by SEQ ID NO:11 is processed to generate the miRNA described by SEQ ID NO:18 (CDPK-152 miRNA) as shown in Table 1. When expressed in the plant the miRNA precursor sequence described by SEQ ID NO: 12 is processed to generate the miRNA described by SEQ ID NO: 19 (inx-3-937 miRNA) as shown in Table 1. When expressed in the plant the miRNA precursor sequence described by SEQ ID NO: 13 is processed to generate the miRNAs described by SEQ ID NO: 20 (opr3-541 miRNA) and SEQ ID NO: 21 (ZF-40 miRNA).

miRNA target sequences are Gm CDPK (SEQ ID NO: 14) (US60/900466), Gm Inx-3 (SEQ ID NO: 15) (U.S. 61/049001) , Gm opr3 (SEQ ID NO: 16) (U.S. 60/900146) and Gm ZF 40 (SEQ ID NO: 17) (U.S. 61/16776).

Plant transformation binary vectors to express the miRNA constructs described by SEQ ID NO: 11-13 were generated using either a soybean cyst nematode (SCN) inducible promoter or a constitutive promoter. For this, the gene fragments described by SEQ ID NO: 11-13 were operably linked to the SCN inducible GmMTN3 promoter (WO 2008/095887), the SCN inducible At trehalose-6-phosphate phosphatase-like promoter (W02008/071726), or the constitutive super promoter (U.S. Pat. No. 5,955,646) as designated in Table 1. The resulting plant binary vectors contain a plant transformation selectable marker consisting of a modified Arabidopsis AHAS gene conferring tolerance to the herbicide Arsenal (BASF Corporation, Florham Park, N.J.).

TABLE 1

| | | | miRNA | Artificial |
|---|---|---|---|---|
| Binary | | | precursor | miRNA |
| Vector | Promoter | | SEQ | SEQ |
| Name | Name | miRNA Name | ID NO: | ID NO: |
| RTP1422-1 | AtTPP | pr-GM pre-miR166h | 11 | 18 |
| | | CDPK-152 | | |
| RTP3178-1 | Super | pr-GM pre-miR166h | 12 | 19 |
| | | Hg inx-3-937 | | |
| RTP4227-4 | GmMTN3 | pr-2x pre-miR | 13 | 20, 21 |
| | | opr3-541; ZF-40 | | |

### Example 6

### Nematode Bioassay

The binary vectors described in Table 1 were used in the rooted plant assay system disclosed in commonly owned copending U.S. Pat. Pub. 2008/0153102. Transgenic roots were generated after transformation with the binary vectors described in Example 1. Multiple transgenic root lines were sub-cultured and inoculated with surface-decontaminated race 3 SCN second stage juveniles (J2) at the level of about 500 J2/well. Four weeks after nematode inoculation, the cyst number in each well was counted. For each transformation construct, the number of cysts per line was calculated to determine the average cyst count and standard error for the construct. The cyst count values for each transformation construct was compared to the cyst count values of an empty vector control tested in parallel to determine if the construct tested results in a reduction in cyst count. Bioassay results of constructs containing the recombinant microRNA precursor molecules represented by SEQ ID NO: 11, 12 or 13 respectively containing the miRNA sequences described by SEQ ID NO: 18, 19, 20 and/or 21 resulted in a general trend of reduced soybean cyst nematode cyst count over many of the lines tested in the designated construct containing a SCN inducible promoter or a constitutive promoter operably linked to each of the miRNAs as described in Table 1.

### Example 7

### Comparison of Efficacy of Two Different Soybean miRNA Precursors in Producing Artificial miRNAs

A. Artificial MiRNAs Expressed from Soybean MicroRNA Precursor pri-miR166h.

The Glycine max precursor sequence for miR166h was used as a template for expressing artificial microRNAs.

Glycine max precursor for miR166h used for producing artificial miRNA

| Sequence name | source | sequence |
|---|---|---|
| pre-miR166h (SEQ ID | Glycine max | tctcaaactcgtttgtgctgagagaaccaagggtttcttcc |
| No. 10) | | cttgcagagaagaaaatttggatacatatggttaagttta |
| | | tttagatatcttgtttgttctttcatctttctcagatatggtttgg |
| | | aaatgggagattggggatgatgggaatgttgtttggctc |
| | | gagaaaaagctttaaaggttggattttgaggctatcccttt |
| | | atgtgatctcggaccaggcttcattcccgtcaaccttatct |
| | | ctctctcttgagatcttctcccatggaggggtgatggcttat |
| | | gtatataggtttcccagctgtagcatctttagggtttga- |
| | | gattcctaaccatctttacttcc |

The 21 nt miR166h having the sequence TCGGACCAGGCTTCATTCCCG is underlined.

| Sequence name | sequence |
|---|---|
| miR-let-7 (SEQ ID | tgaggtagtaggttgtatagt |
| NO. 6) 5′ to 3′ | |
| miR-let-70-143 (SEQ ID | tggatggtcagaaagaagacg |
| NO. 8) 5′ to 3′ | |

Artificial precursor for expressing miR-let-7 from pri-miR166h

To create a sequence that when expressed in soy will produce the let-7 microRNA, sequence pri-miR166h was modified as follows:
1. The sequence
   GATGATGGGAATGTTGTTTGGCTCGAGAAAAAGCTTTAAAGGTTGGATTTTGAGGC TATCCCTTTATGTGATCTCGGACCAGGCTTCATTCCCGTCAA spanning nucleotides 143-240 of pri-mir166h including the miR166h (underlined) and miR166h star (underlined) was replaced with sequence GATGAACTATACATCCTACTACCACAGAAAAAGCTTTAAAGGTTGGATTTTGAGGCT ATCCCTTTATGTGATCTGAGGTAGTAGGTTGTATAGTTCAA containing miR-let-7 (underlined) and miR-let-7 star (underlined).

Artificial precursor for expressing miR-let-70-143 from pri-miR166h

To create a sequence that when expressed in soy will produce the let-70-143 microRNA, sequence pri-miR166h was modified as follows:
1. The sequence
   GATGATGGGAATGTTGTTTGGCTCGAGAAAAAGCTTTAAAGGTTGGATTTTGAGGC TATCCCTTTATGTGATCTCGGACCAGGCTTCATTCCCGTCAA spanning nucleotides 143-240 of pri-mir166h including the miR166h (underlined) and miR166h star (underlined) was replaced with sequence GATGACGTCTTCTATCTGACCATACAGAAAAAGCTTTAAAGGTTGGATTTTGAGGCT ATCCCTTTATGTGATCTGGATGGTCAGAAAGAAGACGTCAA containing miR-let-70-143 (underlined) and miRdet-70-143 star (underlined).

B. Artificial miRNAs Expressed from Soybean MicroRNA Precursor pri-miR-GMmerge_{—}4110158.

The Glycine max precursor sequence for miR-GMmerge_{—}4110158 was identified from our small RNA deep sequencing and subsequent bioinformatics analysis. This precursor was used as a template for expressing artificial microRNAs.

Glycine max precursor for miR-GMmerge_4110158

| Sequence name | source | sequence |
|---|---|---|
| pri-miR- | Glycine max | gtcatggtttttatgtagaaatctctcattaatatgattttcgtt |
| GMmerge_4110158 | | gtttcattgatgatgtaagcaaaattgatcagaattctagt |
| (SEQ ID NO. 22) | | gtatcaggtttacaaaaacaattaccttgtcaatctcaga |
| | | atgaaaaaagtttagagatttaccatctatttaagggaa |
| | | catgtttgttattgactaggctatgctagcaagggaattga |
| | | tagttcttgataatgttgatcaagttggactcctaaagatgt |
| | | ttccaaggagtagagatactttgttacgtgaatgcctagg |
| | | tgaagggggcagaatcatcataatttctagggat |

The 21 nt miR-GMmerge_{—}4110158 having the sequence CTATTTAAGGGAACATGTTTG is underlined.

| Sequence name | sequence |
|---|---|
| miR-let-7 (SEQ ID | tgaggtagtaggttgtatagt |
| NO. 6) 5′ to 3′ | |
| miR-let-70-143(SEQ ID | tggatggtcagaaagaagacg |
| NO. 8) 5′ to 3′ | |

Artificial precursor for expressing miR-let-7 from pri-miR-GMmerge_{—}4110158

To create a sequence that when expressed in soy will produce the let-7 microRNA, sequence pri-miR-GMmerge_{—}4110158 was modified as follows:
1. The sequence
   ACCATCTATTTAAGGGAACATGTTTGTTATTGACTAGGCTATGCTAGCAAGGGAATTGA TAGTTCTTGATAATGTTGATCAAGTTGGACTCCTAAAGATGTTTCCAAGGAGTAGAGATA CTTT spanning nucleotides 148-270 of pri-miR-GMmerge_{—}4110158 including the miR-GMmerge_{—}4110158 (underlined) and miR-GMmerge_{—}4110158 star (underlined) was replaced with sequence ACCATTGAGGTAGTAGGTTGTATAGTTTATTGACTAGGCTATGCTAGCAAGGGAATTGA TAGTTCTTGATAATGTTGATCAAGTTGGACTCCGCTTTACAACTTAGAGGCTTCAAGATA CTTT containing miR-let-7 (underlined) and miR-let-7 star (underlined).

Artificial precursor for expressing miR-let-70-143 from pri-miR-GMmerge_{—}4110158

To create a sequence that when expressed in soy will produce the let-70-143 microRNA, sequence pri-miR-GMmerge_{—}4110158 was modified as follows:
1. The sequence
   ACCATCTATTTAAGGGAACATGTTTGTTATTGACTAGGCTATGCTAGCAAGGGAATTGA TAGTTCTTGATAATGTTGATCAAGTTGGACTCCTAAAGATGTTTCCAAGGAGTAGAGATA CTTT spanning nucleotides 148-270 of pri- miR-GMmerge_{—}4110158 including the miR-GMmerge_{—}4110158 (underlined) and miR-GMmerge_{—}4110158 star (underlined) was replaced with sequence ACCATTCTTCTATCTGACCATACAGATTATTGACTAGGCTATGCTAGCAAGGGAATTGAT AGTTCTTGATAATGTTGATCAAGTTGGACTCCTCTCTATGGTTAGTAGGGGAGAAGATACTTT containing miR-let-70-143 (underlined) and miR-let-70-143 star (underlined).

C. Vector Construction

Four binary vectors, RTP2466, RTP8113, RTP2276 and RTP8111, were constructed in such a way that each miRNA precursor was driven by SuperPromoter and NOS terminator to express the artificial microRNAs (Table 2 and 3). The microRNA precursors were synthesized and cloned into the binary vector using the restriction sites Ascl and Sbfl.

TABLE 2

| | | | | Relative | |
|---|---|---|---|---|---|
| | SEQ | | | miRNA | P- |
| Construct | ID NO | miRNA Precursor | miRNA | Expression | value |
| RTP2466 | 23 | Pri-miR166h | let-7 | 4.39670 | P<0.01 |
| RTP8113 | 24 | pri-miR- | let-7 | 0.01609 | P<0.01 |
| | | GMmerge_4110158 | | | |

TABLE 3

| | SEQ | | | Relative | |
|---|---|---|---|---|---|
| | ID | | | miRNA | P- |
| Construct | NO | miRNA Precursor | miRNA | Expression | value |
| RTP2276 | 26 | pri-miR166h | let-70-143 | 0.13325 | P<0.01 |
| RTP8111 | 25 | pri-miR- | let-70-143 | 0.00019 | P<0.01 |
| | | GMmerge_4110158 | | | |

D. Soybean Root Transformation and Analysis of miRNA Expression

The binary vectors described in Table 2 and 3 were used in the rooted plant assay system disclosed in commonly owned copending U.S. Pat. Pub. 2008/0153102. Multiple transgenic root lines for each construct were sub-cultured and harvested. Total RNAs were extracted with TRI-zol reagent. qRT-PCR based miRNA assays specific to let-7 and let-70-143, respectively, were customized order from Applied Biosystems. The assays were used to determine the expression of artificial miRNAs according to manufactory instruction. Soybean glyceraldehyde-3-phosphate dehydrogenase (GAPDH) was used as an internal control for normalizing miRNA expression. The statistics analysis was applied to determine efficacy of two precursors to express let-7 and let-70-143.

The results indicated that miR166h precursor was able to produce 273-fold higher of let-7 miRNA (P<0.01) and 700-fold higher let-70-143 miRNA (P<0.01) in comparison to GMmerge_{—}4110158 precursor. Thus, soybean miR166h precursor is much better than GMmerge_{—}4110158 precursor in producing artificial miRNAs in soybean.

### DESCRIPTION OF THE FIGURES

FIG. 1

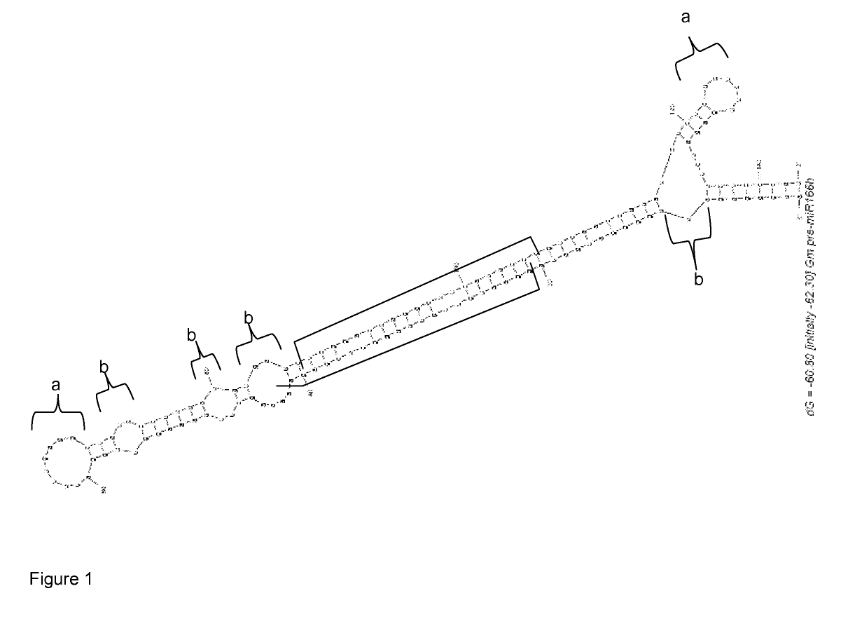

Secondary structure of base pairs 139 to 281 of SEQ ID NO: 1 predicted with mfold (Zuker (2003)), using the following settings: RNA is defined as linear, folding temperature is fixed at 37° C., the ionic conditions are set as 1M NaCl, no divalent ions, percent suboptimality is set to 5, upper bound on the number of computed foldings is set to 50, the window parameter is default, maximum interior/bulge loop size is set to 30, the maximum asymmetry of an interior/bulge loop is set to 30, the maximum distance between paired bases is not limited.

MicroRNA sequence and microRNA star sequence are marked with a frame.
a) loop
b) bulge

1. An isolated nucleic acid molecule comprising a nucleic acid molecule selected from the group consisting of:
I) the nucleic acid molecule of SEQ ID NO: 1;
II) a nucleic acid molecule comprising at least 100 consecutive base pairs (bp) of the nucleic acid sequence of SEQ ID NO: 1;
III) a nucleic acid molecule having at least 70% sequence identity over a sequence of at least 100 consecutive nucleic acid bp to the nucleic acid sequence of SEQ ID NO: 1;
IV) a nucleic acid molecule of at least 100 bp which hybridizes under stringent conditions with at least 100 consecutive bp of the nucleic acid sequence of SEQ ID NO: 1;
V) a nucleic acid molecule able to form a secondary structure homologous to the secondary structure formed by the nucleic acid sequence of SEQ ID NO: 1; and
VI) the complement of any of the sequences as defined in I) to V).

2. The isolated nucleic acid molecule of claim 1 wherein
a) by 225-245 of the nucleic acid sequence of SEQ ID NO: 1, or the corresponding by of the nucleic acid molecules as defined in claim 1 II) to VI), are replaced by a nucleic acid sequence comprising at least 20, 21 or 22 bp, or a multiple thereof, complementary to a target gene;
b) by 159-179 of SEQ NO: 1 the nucleic acid sequence of SEQ ID NO: 1, or the corresponding by of the nucleic acid molecules as defined in claim 1 II) to VI), are replaced by a nucleic acid sequence comprising at least 20, 21 or 22 bp, or a multiple thereof, complementary to a); and
c) a) and b) and a nucleic acid molecule separating a) and b) are able to from a stem loop structure.

3. The isolated nucleic acid of claim 1, wherein by 225-245 of the nucleic acid sequence of SEQ ID NO:1 or the corresponding by of the nucleic acid molecules as defined in claim 1 II) to VI) are replaced by a sequence selected from the group consisting of:
a. the nucleic acid molecule of SEQ ID NO: 18, 19, 20 and/or 21 or a multitude thereof;
b. a nucleic acid molecule having at least 15 consecutive by of the nucleic acid sequence of SEQ ID NO: 18, 19, 20 and/or 21or a multitude thereof;
c. a nucleic acid molecule having at least 70% sequence identity to the entire nucleic acid sequence of SEQ ID NO: 18, 19, 20 and/or 21or a multitude thereof;
d. a nucleic acid molecule comprising 5 mismatches when compared to the nucleic acid sequence of SEQ ID NO: 18, 19, 20 and/or 21 or a multitude thereof; and
e. a nucleic acid molecule which hybridizes under high stringent conditions with the nucleic acid molecule of SEQ ID NO: 18, 19, 20 and/or 21 or a multitude thereof.

4. A plant expression construct comprising the isolated nucleic acid molecule as defined in claim 1.

5. A plant expression vector comprising:
a. the isolated nucleic acid molecule as defined in claim 1; or
b. an expression construct comprising said isolated nucleic acid molecule.

6. A plant, plant cell, or plant seed comprising:
a. the isolated nucleic acid molecule as defined in claim 1;
b. an expression construct comprising said isolated nucleic acid molecule; or
c. a plant expression vector comprising said isolated nucleic acid molecule or said expression construct.

7. A method for modulating expression of a target gene in a plant or part thereof compared to a respective reference plant comprising:
a) functionally linking
i) at least one regulatory nucleic acid molecule functional in a plant with
ii) a recombinant microRNA precursor molecule which is heterologous to the at least one regulatory nucleic acid molecule of i), and which is cleavable in a plant cell to produce said at least one regulatory nucleic acid molecule and
b) introducing the at least one regulatory nucleic acid molecule functionally linked to the recombinant microRNA precursor molecule into a plant or part thereof,
wherein the sequence of the at least one regulatory nucleic acid molecule binds to at least one target sequence in the plant, and
wherein the microRNA precursor molecule is a nucleic acid molecule selected from the group consisting of:
I) the nucleic acid molecule of SEQ ID NO: 1;
II) a nucleic acid molecule comprising at least 100 consecutive base pairs (bp) of the nucleic acid sequence of SEQ ID NO: 1,
III) a nucleic acid molecule having of at least 70% sequence identity to the nucleic acid sequence of SEQ ID NO: 1 over a sequence of at least 100 consecutive nucleic acid bp;
IV) a nucleic acid molecule which hybridizes under stringent conditions with at least 50 consecutive by of the nucleic acid sequence of SEQ ID NO: 1;
V) a nucleic acid molecule able to form a secondary structure homologous to the secondary structure formed by the nucleic acid sequence of SEQ ID NO: 1; and
VI) the complement of any of the sequences as defined in I) to V).

8. The method of claim 7 wherein
a) the at least one regulatory nucleic acid molecule sequence heterologous to the microRNA precursor molecule replaces by 225-245 of the nucleic acid sequence of SEQ ID NO: 1 or the respective by in the nucleic acid molecule sequences as defined in claim 7 II) to VI),
b) at least one regulatory nucleic acid molecule star sequence complementary to the at least one regulatory nucleic acid molecule sequence replaces by 159-179 of the nucleic acid sequence of SEQ ID NO: 1 or the respective by in the nucleic acid molecule sequences as defined in claim 7) II) to VI), and
c) wherein a) and b) and a nucleic acid molecule separating a) and b) are able to form a stem loop structure.

9. The method of claim 7, wherein the at least one regulatory nucleic acid molecule is selected from the group consisting of:
a. the nucleic acid molecule of SEQ ID NO: 18, 19, 20 and/or 21or a multitude thereof;
b. a nucleic acid molecule having at least 15 consecutive base pairs of the nucleic acid sequence of SEQ ID NO: 18, 19, 20 and/or 21or a multitude thereof;
c. a nucleic acid molecule having at least 70% sequence identity to the entire nucleic acid sequence of SEQ ID NO: 18, 19, 20 and/or 21or a multitude thereof;
d. a nucleic acid molecule comprising 5 mismatches when compared to the nucleic acid sequence of SEQ ID NO: 18, 19, 20 and/or 21or a multitude thereof; and
e. a nucleic acid molecule which hybridizes under high stringent conditions with the nucleic acid molecule of SEQ ID NO: 18, 19, 20 and/or 21 or a multitude thereof.

10. The method of claim 7, wherein the at least one regulatory nucleic acid molecule and at least one regulatory nucleic acid molecule star sequence complementary to the regulatory nucleic acid molecule sequence consist of 20, 21 or 22 by or a multiple of 20, 21 or 22 bp.

11. The method of claim 7, wherein the plant is a dicotyledonous plant.

12. (canceled)

13. A process for the production of a transgenic plant comprising the steps of
I. providing an isolated nucleic acid as defined in claim 1, a plant expression construct comprising said isolated nucleic acid and/or a plant expression vector comprising said isolated nucleic acid or said expression construct;
II. introducing said isolated nucleic acid, said plant expression construct, and/or said plant expression vector into a plant cell, plant, or part of a plant; and
III. regenerating a transgenic plant from said plant cell, plant, or part of a plant.

14. The isolated nucleic acid molecule of claim 2, a plant expression construct comprising said isolated nucleic acid molecule, or a plant expression vector comprising said isolated nucleic acid molecule or said expression construct, wherein the bp as defined in claim 2 a) and/or b) are selected from the group consisting of:
a. the nucleic acid molecule of SEQ ID NO: 18, 19, 20 and/or 21;
b. a nucleic acid molecule having at least 15 consecutive base pairs of the nucleic acid sequence of SEQ ID NO: 18, 19, 20 and/or 21;
c. a nucleic acid molecule having at least 70% sequence identity to the entire nucleic acid sequence of SEQ ID NO: 18, 19, 20 and/or 21;
d. a nucleic acid molecule comprising 5 mismatches when compared to the nucleic acid sequence of SEQ ID NO: 18, 19, 20 and/or 21; and
e. a nucleic acid molecule which hybridizes under high stringent conditions with the nucleic acid molecule of SEQ ID NO: 18, 19, 20 and/or 21.

15. A method of conferring nematode resistance to a plant, said method comprising the steps of:
I. preparing an isolated nucleic acid molecule as defined in claim 3, an expression construct comprising said isolated nucleic acid molecule, or an expression vector comprising said isolated nucleic acid molecule or said expression construct;
II. transforming a recipient plant with said isolated nucleic acid molecule, expression construct or expression vector;
III. producing one or more transgenic offspring of said recipient plant; and
IV. selecting the offspring for nematode resistance.

16. The method of claim 15, wherein the isolated nucleic acid molecule comprises a nucleic acid sequence selected from the group consisting of the nucleic acid sequence of SEQ ID NO: 11, 12 and 13.

17. The method of claim 7, wherein the plant is of the genus Glycine.


SEQUENCE LISTING (26 sequences)

SEQ ID NO: 1 - DNA, 793 bp, Glycine max
gatttcgtctctcaaactcgtttgtgctgagagaaccaagggtttcttcccttgcagagaagaaaatttggatacatatggttaagtttatttagatatcttgtttgttctttcatctttctcagatatggtttggaaatgggagattggggatgatgggaatgttgtttggctcgagaaaaagctttaaaggttggattttgaggctatccctttatgtgatctcggaccaggcttcattcccgtcaaccttatctctctctcttgagatcttctcccatggaggggtgatggcttatgtatataggtttcccagctgtagcatctttagggtttgagattcctaaccatctttacttcctgtcaagtttcagtccatgtggttggcttcattttttcagcccaggtgaatagagaaaagtgctgagaagatgcatgaagttaagatgaattaatggatctaaaacataacaaaattggtggcacacaatgcatacagaattttcctttcatttgttggttatcacttcatcgtttttcttgcttgtttctaccaaaataaaaggcgaaagagggagtgttgtggtggtggttgggctagcctgaaaaattgtaattcctaccaaggttttaaaaaatatcagtaacaggaatttcaatcacaatatcaagctttgtgggcgattgcatcttgtggtgacatcatcggtcatatttttccacaatgtcaggaatcacaaccacactttaaaacctttatacaattttcctaaacaaaaaaaaaaaaaaaaaa

SEQ ID NO: 2 - RNA, 21 nt, Glycine max
ucggaccaggcuucauucccc

SEQ ID NO: 3 - DNA, 1018 bp, Glycine max
tctcaaactcgtttgtgctgagagaaccaagggtttcttcccttgcagagaagaaaatttggatacatatggttaagtttatttagatatcttgtttgttctttcatctttctcagatatggtttggaaatgggagattggggatgatgggaatgttgtttggctcgaggtaactgcatggtcttaattttgttcatcttttgaagctttaatttatttatgggtttcaatcttttttgatcccttgaaacagaaaaagctttaaaggttggattttgaggctatccctttatgtgatctcggaccaggcttcattcccgtcaaccttatctctctctcttgagatcttctcccatggaggggtgatggcttatggtaattaattaaggcgatgtcaaaacaagactcttgtgatagatatatctggtcaatgatgacattaaaactctcttgtttgttattttttatcggtaaatatgttacttgttaaaatattagttttgttattaagattgatctgtaattttttttcttaaccatccaatctattatatatctcctatacttatttgttttgtttgtgtttgtaaattcagacattgcatgggacaaaacttgaaacatttgtcaagaactctattgaacaagatgcctttgaccttgttgtaattggtgctttgttttttgtcatctttcttatggctccattatactttatatatcttcatttctttcaacatctctctcgatcgtgagttaatttaacggttgagacttgagatttgtgctattatatattatgtctatctaaaaggatcaggctccaaaatctttatatagtatataattatttttcttggattatgattagtgagtaattggtattaacttttttaagttaaatattctgcgttgagaaacccaaggatctttgaaattctgtattttgtcagtatataggtttcccagctgtagcatctttagggtttgagattcctaaccatctttacttcctgtc

SEQ ID NO: 4 - DNA, 21 bp, Artificial
  encode microRNA
tcatatgtgttcttcagttat

SEQ ID NO: 5 - DNA, 21 bp, Artificial
  encode microRNA star
aactgacgaacacatatgaga

SEQ ID NO: 6 - DNA, 21 bp, Caenorhabditis elegans
tgaggtagtaggttgtatagt

SEQ ID NO: 7 - DNA, 718 bp, Artificial
  stacking miRNA precursors
tctcaaactcgtttgtgctgagagaaccaagggtttcttcccttgcagagaagaaaatttggatacatatggttaagtttatttagatatcttgtttgttctttcatctttctcagatatggtttggaaatgggagattggggatgaactatacatcctactaccacagaaaaagctttaaaggttggattttgaggctatccctttatgtgatctgaggtagtaggttgtatagttcaaccttatctctctctcttgagatcttctcccatggaggggtgatggcttatgtatataggtttcccagctgtagcatctttagggtttgagattcctaaccatctttacttcctgtatttaaattctcaaactcgtttgtgctgagagaaccaagggtttcttcccttgcagagaagaaaatttggatacatatggttaagtttatttagatatcttgtttgttctttcatctttctcagatatggtttggaaatgggagattggggatgacgtcttctatctgaccatacagaaaaagctttaaaggttggattttgaggctatccctttatgtgatctggatggtcagaaagaagacgtcaaccttatctctctctcttgagatcttctcccatggaggggtgatggcttatgtatataggtttcccagctgtagcatctttagggtttgagattcctaaccatctttacttcctgt

SEQ ID NO: 8 - DNA, 21 bp, Artificial
  encode artificial miRNA
tggatggtcagaaagaagacg

SEQ ID NO: 9 - DNA, 398 bp, Artificial
  stacking miRNA cassette
tatggtttggaaatgggagattggggatgaactatacatcctactaccacagaaaaagctttaaaggttggattttgaggctatccctttatgtgatctgaggtagtaggttgtatagttcaaccttatctctctctcttgagatcttctcccatggaggggtgatggcttatgtatataggtttcccagctgtaatttaaattatggtttggaaatgggagattggggatgaactcttctatctgaccatacagaaaaagctttaaaggttggattttgaggctatccctttatgtgatctggatggtcagaaagaagacgtcaaccttatctctctctcttgagatcttctcccatggaggggtgatggcttatgtatataggtttcccagctgta

SEQ ID NO: 10 - DNA, 355 bp, Glycine max
tctcaaactcgtttgtgctgagagaaccaagggtttcttcccttgcagagaagaaaatttggatacatatggttaagtttatttagatatcttgtttgttctttcatctttctcagatatggtttggaaatgggagattggggatgatgggaatgttgtttggctcgagaaaaagctttaaaggttggattttgaggctatccctttatgtgatctcggaccaggcttcattcccgtcaaccttatctctctctcttgagatcttctcccatggaggggtgatggcttatgtatataggtttcccagctgtagcatctttagggtttgagattcctaaccatctttacttcctgt

SEQ ID NO: 11 - DNA, 352 bp, Artificial
  miRNA precursor
tctcaaactcgtttgtgctgagagaaccaagggtttcttcccttgcagagaagaaaatttggatacatatggttaagtttatttagatatcttgtttgttctttcatctttctcagatatggtttggaaatgggagattggggatgactagagattttgcaagcgttagaaaaagctttaaaggttggattttgaggctatccctttatgtgatctatcgcttgcaatatctctagtcaaccttatctctctctcttgagatcttctcccatggaggggtgatggcttatgtatataggtttcccagctgtagcatctttagggtttgagattcctaaccatctttacttcc

SEQ ID NO: 12 - DNA, 718 bp, Artificial
  miRNA precursor
tctcaaactcgtttgtgctgagagaaccaagggtttcttcccttgcagagaagaaaatttggatacatatggttaagtttatttagatatcttgtttgttctttcatctttctcagatatggtttggaaatgggagattggggatgatcgcaaaggtgaagcacaagagaaaaagctttaaaggttggattttgaggctatccctttatgtgatctcatgtgcttcagctttgcgatcaaccttatctctctctcttgagatcttctcccatggaggggtgatggcttatgtatataggtttcccagctgtagcatctttagggtttgagattcctaaccatctttacttcctgtatttaaattctcaaactcgtttgtgctgagagaaccaagggtttcttcccttgcagagaagaaaatttggatacatatggttaagtttatttagatatcttgtttgttctttcatctttctcagatatggtttggaaatgggagattggggatgactagagattttgcaagcgttagaaaaagctttaaaggttggattttgaggctatccctttatgtgatctatcgcttgcaatatctctagtcaaccttatctctctctcttgagatcttctcccatggaggggtgatggcttatgtatataggtttcccagctgtagcatctttagggtttgagattcctaaccatctttacttcctgt

SEQ ID NO: 13 - DNA, 718 bp, Artificial
  miRNA precursor
tctcaaactcgtttgtgctgagagaaccaagggtttcttcccttgcagagaagaaaatttggatacatatggttaagtttatttagatatcttgtttgttctttcatctttctcagatatggtttggaaatgggagattggggatgaagccaagctgctattaatccagaaaaagctttaaaggttggattttgaggctatccctttatgtgatctgcattaatagctgcttggcttcaaccttatctctctctcttgagatcttctcccatggaggggtgatggcttatgtatataggtttcccagctgtagcatctttagggtttgagattcctaaccatctttacttcctgtatttaaattctcaaactcgtttgtgctgagagaaccaagggtttcttcccttgcagagaagaaaatttggatacatatggttaagtttatttagatatcttgtttgttctttcatctttctcagatatggtttggaaatgggagattggggatgacgtccaatatctgttgatacagaaaaagctttaaaggttggattttgaggctatccctttatgtgatctgaatcaacagacattggacgtcaaccttatctctctctcttgagatcttctcccatggaggggtgatggcttatgtatataggtttcccagctgtagcatctttagggtttgagattcctaaccatctttacttcctgt

SEQ ID NO: 14 - DNA, 1045 bp, Glycine max
aagatcctcgtgcgagatatactgctgctcaggctctttcacatccatgggttagagaaggaggagaggcattagagattcctattgatatatctgtcctgaacaacatgcgacagtttgtgaaatatagtcggttgaaacaatttgcactaagggcattggctagcacacttaatgaaggagagttgtctgatctaaaagatcagtttgatgcaatagatgtggacaaaaatggttctattagtcttgaggagatgagacaggctcttgctaaagatcaaccttggaagttgaaagaatcacgtgtgctagagatattgcaagcgatagacagcaacacagatgggctagtggatttcaccgagtttgtggcagctactttacatgtacatcaattggaggaacatgattctgacaagtggcagcaacggtcacaggctgcttttgagaaatttgacttggataaggatggctatattactccagatgaacttagaatgcatacgggtttgagaggctccattgatccattgcttgaggaagccgatattgataaagatgggaaaatcagcttaccagaatttcgtagacttctaagaactgcaagcatgggttctcgaacagtaatgagcccaagtcaccgtcatcatcgaaagatttagattagatatgtttcggagacagggataattcaggagatgagagtgatggatttccttttaagatgatgatctcttatgcagattgtgctgaagccctccttgtgatatctattctgctgcacattttgcactctgaccatcattatatggaactctgaaactagagttaatattgactatggcataccacacaggattgtgtgttaagcttagtctagacgaccatgtcaagaggaatttaaatagtgtgtggattgacttgaggtatcagagtttcatcggtgtacatcattggcttgtttgtgccatacatgtaatgtgtaatactgtaactataaataaaaaacttcttactccccttcccctgcaaaaaaaaaaaaaaa

SEQ ID NO: 15 - DNA, 1844 bp, Glycine max
ggacactgacatggactgaaggagtagaaaggtttaattacccaagtttgagcgccgctgtcactggcacagagccgctgacccgtacggcggccgctgcaacagcgggcgccaccaccgacatgttcttccacgcgacgctggcgcgcactttcatttccgcgctgcgcgttcgcggcgacgacgatgtggtggaccgtctaaactactactacacgccgatcatgttggccatcgcgtgccttgtcgtgtccgccaagcagttcggcggctcgcccatcgaatgttgggtgaacccgcactcgcgcgaaagcatggaggagtacatagaggcattctgctggatccagaacacttattgggtgccaatgtatgagcacattccggacagtcatgaagcgcgagaaggacagcaaattggctattaccaatgggtgccgttcattctgatcgcccaagcactgatgttctctttgccatgcattctgtggcgattgctcaattggcaaaatggcaccaacattcagcaactgatttcggctgcttgtgaggcgcgttcagtgatcgacgcggatgagagggaacgcgtggtgggcgcggtggcgcggacattcgtcgaaatgttggacctgcgcgaaattcaaaatcggccccacccttacgcttcatccctcgcccgtttcaacccaattcggctgatgaatggccatttggtctgttccctgtatttgttcaccaaagtgtgctattccgtcaacattatgctccaatttgctcttctcaatgccgcactgacctcaaaagaccattttctgtttgggtttcaagttctgtccgacctttacgagggaaaaccgtggacaaagtcgggccattttccacgagtgactctctgtgacttcgaagtgcgttatttggccaatttgaacagatacactgtacaatgcgctctgatcattaacattatcaacgaaaaggtgttcgctttcttttggctttggtactgtctgcttttatgtgccacaacctgttccgccctgttttggctcagcaacattctgttgcacatcgcccgggtggactacgtgctgaagtttatgcaaattgctgaacacagtgaacaacagcgaagcagcggcagaacgccaaaactatcgcaacaaaagtgggcgatggtggaggagggcgaaatgccacaattcaccaaaagaccatttcgggtgccgagtgcgcattcggtggacaaatttgtggacgaattcctcaaatcggacgggctgttcattctccgattggtggccacaaatgcgggcgaattggtcgtcgttgacattgtcaagtgcctttggagagagttttcttcccgtcagtttcatattcgtccgttggtctatgaaaacgaactgagcgaagaacggaggcgcgaggatgaagacagtcaccacagccttctgctgaatgtgtacagcagcagagggaacggaccgacccatcaacagcagcagaggaagcaaagccaacaacttcgatattccactaatggcaacagcctcggccttccactcccgacaatgtctcggccgccttctgtggtcgcattggacgattctgtcggcacaccgtcgcccgtctgatgaggaggagaaagaagcaaatggccgaagacattctcaacgaatggatgcgatgccatcagattaagaacaacattattattttaatgcgaccttttcacattttgtacataaatgtaattaaatacaatgtcagtcaaattgttaaagataataatttcaatttgaatataaagtgattggttgtcaaaaaaaaaaaaaaaaaaaaaa

SEQ ID NO: 16 - DNA, 1442 bp, Glycine max
aacatatctccctcttcttaattcaacaatggcagataactcaattagcttattttctccatacaacaagatgggcaaattcaacctctctcatagggtggtattggctcccatgaccagatgcagagcgctcaatgggactccactggcagcacatgctgaatactacgctcagagatcaacaccgggtggatttctcatcactgaaggcaccttgatctctccaacttcttctgggtttcctcatgttcctggaatatactcagatgaacaggtagaggcatggagaaatgtagtggacgccgtgcatgccaacggcagctttatcttctgtcaactctggcatgttggccgtgcatcacatccagtgtatcagcctggtggggctctaccctcttcgtccaccagcaaacccatatcagacaagtggaaaattctcatgcccgatggctcccatggcatctatccagagcctcgtgcacttaccacttctgagatatctgaaatagtgcatcattatcgccaagcagctattaatgcaattcgagcaggttttgatggaatcgagattcatggagcacatgggtatctcattgatcaattcttaaaggatgcaatcaatgatagaacagatgaatacggtggaccactagaaaaccggtgcaggttcttaatggaggtagttgaagctgttgtctctgccattggagcggaaagagttgctatcagaatttcaccagcaattgatttcaatgacgcctttgactctgacccacttgggctaggcttagcagtgattgaaagactcaacaatttgcagaaacaagtgggcacaaaactcgcttatcttcatgttactcagcctcgattcacacttttggcgcaaaccgagtcagtgagtgaaaaggaggaagctcatttcatgcagaaatggagagaggcttatgagggaacattcatgtgtagtggagcttttactagggactcaggaatggaagctgtagctgaaggccatgctgatttggtatcctatggtcgtcttttcatctccaatccagacttggttttaaggcttaagctcaatgcacctcttaccaagtataacaggaacacattttacacccaagatcctgttataggctacacagattatcctttctttaatggaacaactgagacaaaattaagtaactagctaaggccatgcatgccctttaattttaatctccatatggctttttgaataataatgttcataacattcaaaactcttcagttgagtttatcctcagacaaacaaattaagtggttcattcacttgttagggtatttagatcttaggttaattagtctccggcattttgatttcatttcaatttgtattcagtctttcattttgaataaaataatattaagttttttgccttaaaaaaaaaaaaaaa

SEQ ID NO: 17 - DNA, 1314 bp, Glycine max
aagaaatttccgaaagtgtgtgtgggatgagaagatgatgaggaaacattggtagaagaaattggagagagagagagagagagagggttggattggttttcttcttcctcttcctaaagagaatctaatctcaatcctcattacacacatatctacagatttcttcattcctctctcaatcttttctgttttcttttcccctctttgacatcctcgttttcgccgaaacaaaaacaactaagattttgttttttgttgtatttttcttcctgttgttatttgactcggttgtctgaatcagttgttgcagcagggtcgggagccacagctatgcagagccaagttgtgtgcaatggttgtaggagccttctgctttacccaagaggagcaaccaatgtctgttgtgcattgtgcaacacaattacctctgttcctccacctgggatggaaatgtctcaactttattgtggaggttgtaggacattgctaatgtacacacgtggagctacaagtgtgagatgttcctgctgtcacactgtaaaccttgttccaccagcatctaatcaagtggctcatgtccattgtgggaactgccggacaacactcatgtatccttatggagctccctcagtcaaatgtgctctttgtcactttattactaatacaaacaatggaaggcttccaatccctgtccatagacccaatgggacaaacaatgctggaacattaccttctacatcaacatcaatgccccaatctcaaagtcaaacggtagtggtagaaaatccaatgtctgttgattcaagtgggaaattggtgagcaatgttgttgttggcgttacaacggataagaaataacatcatcacatataaaaggtacagttcacgtcatgacgcatcaccacagttgctgtcacaggaagatattgtttgtgtatatgaatatatatatgatattgcagcctgcaatggtttaattgaaatcaatatttttccaatataagagttggacgaatatcatgtatgtatgaatgtatatgaggcggacaagtcgtgaaaaggccataaaacctgttttgtgtgattgccagagcctactttgttttttgcttggtacccatacccaagtcaattggttcattcggataaaatatgttgacttccaagtcctagactagtaaatgctactatttctttcatggcttgtgaagcttgtgcttttcttggtgatctgttagttacatctgttgtaatatttgatcttgtgcaagcattgcttcataaaatgactgatatgataaatttttaccagtc

SEQ ID NO: 18 - RNA, 21 nt, Artificial Sequence
  artificial miRNA
uaucgcuugcaauaucucuag

SEQ ID NO: 19 - RNA, 21 nt, Artificial Sequence
  artificial miRNA
ugaucagagcgcauuguacag

SEQ ID NO: 20 - RNA, 21 nt, Artificial Sequence
  artificial miRNA
ugcauuaauagcugcuuggcu

SEQ ID NO: 21 - RNA, 21 nt, Artificial Sequence
  artificial miRNA
ugaaucaacagacauuggacg

SEQ ID NO: 22 - DNA, 322 bp, Glycine max
gtcatggtttttatgtagaaatctctcattaatatgattttcgttgtttcattgatgatgtaagcaaaattgatcagaattctagtgtatcaggtttacaaaaacaattaccttgtcaatctcagaatgaaaaaagtttagagatttaccatctatttaagggaacatgtttgttattgactaggctatgctagcaagggaattgatagttcttgataatgttgatcaagttggactcctaaagatgtttccaaggagtagagatactttgttacgtgaatgcctaggtgaagggggcagaatcatcataatttctagggat

SEQ ID NO: 23 - DNA, 10888 bp, Artificial
  Vector comprising artificial microRNA precursor
tcgaggcgcgcctctcaaactcgtttgtgctgagagaaccaagggtttcttcccttgcagagaagaaaatttggatacatatggttaagtttatttagatatcttgtttgttctttcatctttctcagatatggtttggaaatgggagattggggatgaactatacatcctactaccacagaaaaagctttaaaggttggattttgaggctatccctttatgtgatctgaggtagtaggttgtatagttcaaccttatctctctctcttgagatcttctcccatggaggggtgatggcttatgtatataggtttcccagctgtagcatctttagggtttgagattcctaaccatctttacttcccctgcaggcctaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggccggccgtttaaacttagttactaatcagtgatcagattgtcgtttcccgccttcactttaaactatcagtgtttgacaggatatattggcgggtaaacctaagagaaaagagcgtttattagaataatcggatatttaaaagggcgtgaaaaggtttatccgttcgtccatttgtatgtcaatattgggggggggggaaagccacgttgtgtctcaaaatctctgatgttacattgcacaagataaaaatatatcatcatgaacaataaaactgtctgcttacataaacagtaatacaaggggtgttcgccaccatgagccatatccagcgtgaaacctcgtgctcccgcccgcgcctcaattccaatatggatgccgacctttatggctacaagtgggcgcgcgacaacgtcggccagtcgggcgcgaccatttatcggctttatggcaaacccgatgccccggaactgttcctgaagcacggcaaaggcagcgtcgcaaacgatgtcaccgatgagatggtccgcctgaactggcttaccgagttcatgccgctgccgacgattaagcatttcatccgtaccccggacgatgcctggctcttgaccacggccattccgggcaaaacggcctttcaggtccttgaagagtacccggactccggtgagaatatcgtggacgccctcgcggtcttcctccgccgtttgcatagcatccccgtgtgcaactgccccttcaactcggaccgggttttccgcctggcacaggcccagtcgcgcatgaataacggcctcgttgacgcgagcgatttcgacgatgaacggaatggctggccggtggaacaggtttggaaggaaatgcacaaactgcttccgttctcgccggattcggtggtcacgcatggtgatttttccctggataatctgatctttgacgagggcaagctgatcggctgcatcgacgtgggtcgcgtcggtatcgccgaccgctatcaggacctggcgatcttgtggaattgcctcggcgagttctcgccctcgctccagaagcgcctgttccagaagtacggcatcgacaacccggatatgaacaagctccagttccacctcatgctggacgaatttttttgaacagaattggttaattggttgtaacactggcagagcattacgctgacttgacgggacggcggctttgttgaataaatcgaacttttgctgagttgaaggatcgatgagttgaaggaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccggggtcgatcaaaaagtaatcggggtgaaccgtcagcacgtccgggttcttgccttctgtgatctcgcggtacatccaatcagcaagctcgatctcgatgtactccggccgcccggtttcgctctttacgatcttgtagcggctaatcaaggcttcaccctcggataccgtcaccaggcggccgttcttggccttcttggtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggcggggcctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccccttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccaaatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagatgatcagtgattttgtgccgagctgccggtcggggagctgttggctggctggtggcaggatatattgtggtgtaaacaaattgacgcttagacaacttaataacacattgcggacgtctttaatgtactgaatttagttactgatcactgattaagtactgatatcggtaccaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagctgaaccatggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtacatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaatgggaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgaatggtggcactttcaacgatgtcataacggaaggagatggccggattaaatactgagagatgaaaccggtgattatcagaaccttttatggtctttgtatgcatatggtaaaaaaacttagtttgcaatttcctgtttgttttggtaatttgagtttcttttagttgttgatctgcctgctttttggtttacgtcagactactactgctgttgttgtttggtttcctttctttcattttataaataaataatccggttcggtttactccttgtgactggctcagtttggttattgcgaaatgcgaatggtaaattgagtaattgaaattcgttattagggttctaagctgttttaacagtcactgggttaatatctctcgaatcttgcatggaaaatgctcttaccattggtttttaattgaaatgtgctcatatgggccgtggtttccaaattaaataaaactacgatgtcatcgagaagtaaaatcaactgtgtccacattatcagttttgtgtatacgatgaaatagggtaattcaaaatctagcttgatatgccttttggttcattttaaccttctgtaaacattttttcagattttgaacaagtaaatccaaaaaaaaaaaaaaaaaatctcaactcaacactaaattattttaatgtataaaagatgcttaaaacatttggcttaaaagaaagaagctaaaaacatagagaactcttgtaaattgaagtatgaaaatatactgaattgggtattatatgaatttttctgatttaggattcacatgatccaaaaaggaaatccagaagcactaatcagacattggaagtaggaatatttcaaaaagttttttttttttaagtaagtgacaaaagcttttaaaaaatagaaaagaaactagtattaaagttgtaaatttaataaacaaaagaaattttttatattttttcatttctttttccagcatgaggttatgatggcaggatgtggatttcatttttttccttttgatagccttttaattgatctattataattgacgaaaaaatattagttaattatagatatattttaggtagtattagcaatttacacttccaaaagactatgtaagttgtaaatatgatgcgttgatctcttcatcattcaatggttagtcaaaaaaataaaagcttaactagtaaactaaagtagtcaaaaattgtactttagtttaaaatattacatgaataatccaaaacgacatttatgtgaaacaaaaacaatatctagtacgcgtcaattgatttaaatttaattaatcgactctagattcgacggtatcgataagctcgcggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccgcgagctggtcaatcccattgcttttgaagcagctcaacattgatctctttctcgatcgagggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaa

SEQ ID NO: 24 - DNA, 10858 bp, artificial
  Vector comprising artificial microRNA precursor
gtgattttgtgccgagctgccggtcggggagctgttggctggctggtggcaggatatattgtggtgtaaacaaattgacgcttagacaacttaataacacattgcggacgtctttaatgtactgaatttagttactgatcactgattaagtactgatatcggtaccaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagctgaaccatggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtacatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaatgggaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgaatggtggcactttcaacgatgtcataacggaaggagatggccggattaaatactgagagatgaaaccggtgattatcagaaccttttatggtctttgtatgcatatggtaaaaaaacttagtttgcaatttcctgtttgttttggtaatttgagtttcttttagttgttgatctgcctgctttttggtttacgtcagactactactgctgttgttgtttggtttcctttctttcattttataaataaataatccggttcggtttactccttgtgactggctcagtttggttattgcgaaatgcgaatggtaaattgagtaattgaaattcgttattagggttctaagctgttttaacagtcactgggttaatatctctcgaatcttgcatggaaaatgctcttaccattggtttttaattgaaatgtgctcatatgggccgtggtttccaaattaaataaaactacgatgtcatcgagaagtaaaatcaactgtgtccacattatcagttttgtgtatacgatgaaatagggtaattcaaaatctagcttgatatgccttttggttcattttaaccttctgtaaacattttttcagattttgaacaagtaaatccaaaaaaaaaaaaaaaaaatctcaactcaacactaaattattttaatgtataaaagatgcttaaaacatttggcttaaaagaaagaagctaaaaacatagagaactcttgtaaattgaagtatgaaaatatactgaattgggtattatatgaatttttctgatttaggattcacatgatccaaaaaggaaatccagaagcactaatcagacattggaagtaggaatatttcaaaaagttttttttttttaagtaagtgacaaaagcttttaaaaaatagaaaagaaactagtattaaagttgtaaatttaataaacaaaagaaattttttatattttttcatttctttttccagcatgaggttatgatggcaggatgtggatttcatttttttccttttgatagccttttaattgatctattataattgacgaaaaaatattagttaattatagatatattttaggtagtattagcaatttacacttccaaaagactatgtaagttgtaaatatgatgcgttgatctcttcatcattcaatggttagtcaaaaaaataaaagcttaactagtaaactaaagtagtcaaaaattgtactttagtttaaaatattacatgaataatccaaaacgacatttatgtgaaacaaaaacaatatctagtacgcgtcaattgatttaaatttaattaatcgactctagattcgacggtatcgataagctcgcggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccgcgagctggtcaatcccattgcttttgaagcagctcaacattgatctctttctcgatcgagggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcgaggcgcgccgtcatggtttttatgtagaaatctctcattaatatgattttcgttgtttcattgatgatgtaagcaaaattgatcagaattctagtgtatcaggtttacaaaaacaattaccttgtcaatctcagaatgaaaaaagtttagagatttaccattgaggtagtaggttgtatagtttattgactaggctatgctagcaagggaattgatagttcttgataatgttgatcaagttggactccgctttacaacttagaggcttcaagatactttgttacgtgaatgcctaggtgaagggggcagaatcatcataatttctagggatcctgcaggcctaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggccggccgtttaaacttagttactaatcagtgatcagattgtcgtttcccgccttcactttaaactatcagtgtttgacaggatatattggcgggtaaacctaagagaaaagagcgtttattagaataatcggatatttaaaagggcgtgaaaaggtttatccgttcgtccatttgtatgtcaatattgggggggggggaaagccacgttgtgtctcaaaatctctgatgttacattgcacaagataaaaatatatcatcatgaacaataaaactgtctgcttacataaacagtaatacaaggggtgttcgccaccatgagccatatccagcgtgaaacctcgtgctcccgcccgcgcctcaattccaatatggatgccgacctttatggctacaagtgggcgcgcgacaacgtcggccagtcgggcgcgaccatttatcggctttatggcaaacccgatgccccggaactgttcctgaagcacggcaaaggcagcgtcgcaaacgatgtcaccgatgagatggtccgcctgaactggcttaccgagttcatgccgctgccgacgattaagcatttcatccgtaccccggacgatgcctggctcttgaccacggccattccgggcaaaacggcctttcaggtccttgaagagtacccggactccggtgagaatatcgtggacgccctcgcggtcttcctccgccgtttgcatagcatccccgtgtgcaactgccccttcaactcggaccgggttttccgcctggcacaggcccagtcgcgcatgaataacggcctcgttgacgcgagcgatttcgacgatgaacggaatggctggccggtggaacaggtttggaaggaaatgcacaaactgcttccgttctcgccggattcggtggtcacgcatggtgatttttccctggataatctgatctttgacgagggcaagctgatcggctgcatcgacgtgggtcgcgtcggtatcgccgaccgctatcaggacctggcgatcttgtggaattgcctcggcgagttctcgccctcgctccagaagcgcctgttccagaagtacggcatcgacaacccggatatgaacaagctccagttccacctcatgctggacgaatttttttgaacagaattggttaattggttgtaacactggcagagcattacgctgacttgacgggacggcggctttgttgaataaatcgaacttttgctgagttgaaggatcgatgagttgaaggaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccggggtcgatcaaaaagtaatcggggtgaaccgtcagcacgtccgggttcttgccttctgtgatctcgcggtacatccaatcagcaagctcgatctcgatgtactccggccgcccggtttcgctctttacgatcttgtagcggctaatcaaggcttcaccctcggataccgtcaccaggcggccgttcttggccttcttggtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggcggggcctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccccttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccaaatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagatgatca

SEQ ID NO: 25 - DNA, 10858 bp, artificial
  Vector comprising artificial microRNA precursor
tccagaagcactaatcagacattggaagtaggaatatttcaaaaagttttttttttttaagtaagtgacaaaagcttttaaaaaatagaaaagaaactagtattaaagttgtaaatttaataaacaaaagaaattttttatattttttcatttctttttccagcatgaggttatgatggcaggatgtggatttcatttttttccttttgatagccttttaattgatctattataattgacgaaaaaatattagttaattatagatatattttaggtagtattagcaatttacacttccaaaagactatgtaagttgtaaatatgatgcgttgatctcttcatcattcaatggttagtcaaaaaaataaaagcttaactagtaaactaaagtagtcaaaaattgtactttagtttaaaatattacatgaataatccaaaacgacatttatgtgaaacaaaaacaatatctagtacgcgtcaattgatttaaatttaattaatcgactctagattcgacggtatcgataagctcgcggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccgcgagctggtcaatcccattgcttttgaagcagctcaacattgatctctttctcgatcgagggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcgaggcgcgccgtcatggtttttatgtagaaatctctcattaatatgattttcgttgtttcattgatgatgtaagcaaaattgatcagaattctagtgtatcaggtttacaaaaacaattaccttgtcaatctcagaatgaaaaaagtttagagatttaccattcttctatctgaccatacagattattgactaggctatgctagcaagggaattgatagttcttgataatgttgatcaagttggactcctctctatggttagtaggggagaagatactttgttacgtgaatgcctaggtgaagggggcagaatcatcataatttctagggatcctgcaggcctaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggccggccgtttaaacttagttactaatcagtgatcagattgtcgtttcccgccttcactttaaactatcagtgtttgacaggatatattggcgggtaaacctaagagaaaagagcgtttattagaataatcggatatttaaaagggcgtgaaaaggtttatccgttcgtccatttgtatgtcaatattgggggggggggaaagccacgttgtgtctcaaaatctctgatgttacattgcacaagataaaaatatatcatcatgaacaataaaactgtctgcttacataaacagtaatacaaggggtgttcgccaccatgagccatatccagcgtgaaacctcgtgctcccgcccgcgcctcaattccaatatggatgccgacctttatggctacaagtgggcgcgcgacaacgtcggccagtcgggcgcgaccatttatcggctttatggcaaacccgatgccccggaactgttcctgaagcacggcaaaggcagcgtcgcaaacgatgtcaccgatgagatggtccgcctgaactggcttaccgagttcatgccgctgccgacgattaagcatttcatccgtaccccggacgatgcctggctcttgaccacggccattccgggcaaaacggcctttcaggtccttgaagagtacccggactccggtgagaatatcgtggacgccctcgcggtcttcctccgccgtttgcatagcatccccgtgtgcaactgccccttcaactcggaccgggttttccgcctggcacaggcccagtcgcgcatgaataacggcctcgttgacgcgagcgatttcgacgatgaacggaatggctggccggtggaacaggtttggaaggaaatgcacaaactgcttccgttctcgccggattcggtggtcacgcatggtgatttttccctggataatctgatctttgacgagggcaagctgatcggctgcatcgacgtgggtcgcgtcggtatcgccgaccgctatcaggacctggcgatcttgtggaattgcctcggcgagttctcgccctcgctccagaagcgcctgttccagaagtacggcatcgacaacccggatatgaacaagctccagttccacctcatgctggacgaatttttttgaacagaattggttaattggttgtaacactggcagagcattacgctgacttgacgggacggcggctttgttgaataaatcgaacttttgctgagttgaaggatcgatgagttgaaggaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccggggtcgatcaaaaagtaatcggggtgaaccgtcagcacgtccgggttcttgccttctgtgatctcgcggtacatccaatcagcaagctcgatctcgatgtactccggccgcccggtttcgctctttacgatcttgtagcggctaatcaaggcttcaccctcggataccgtcaccaggcggccgttcttggccttcttggtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggcggggcctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccccttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccaaatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagatgatcagtgattttgtgccgagctgccggtcggggagctgttggctggctggtggcaggatatattgtggtgtaaacaaattgacgcttagacaacttaataacacattgcggacgtctttaatgtactgaatttagttactgatcactgattaagtactgatatcggtaccaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagctgaaccatggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtacatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaatgggaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgaatggtggcactttcaacgatgtcataacggaaggagatggccggattaaatactgagagatgaaaccggtgattatcagaaccttttatggtctttgtatgcatatggtaaaaaaacttagtttgcaatttcctgtttgttttggtaatttgagtttcttttagttgttgatctgcctgctttttggtttacgtcagactactactgctgttgttgtttggtttcctttctttcattttataaataaataatccggttcggtttactccttgtgactggctcagtttggttattgcgaaatgcgaatggtaaattgagtaattgaaattcgttattagggttctaagctgttttaacagtcactgggttaatatctctcgaatcttgcatggaaaatgctcttaccattggtttttaattgaaatgtgctcatatgggccgtggtttccaaattaaataaaactacgatgtcatcgagaagtaaaatcaactgtgtccacattatcagttttgtgtatacgatgaaatagggtaattcaaaatctagcttgatatgccttttggttcattttaaccttctgtaaacattttttcagattttgaacaagtaaatccaaaaaaaaaaaaaaaaaatctcaactcaacactaaattattttaatgtataaaagatgcttaaaacatttggcttaaaagaaagaagctaaaaacatagagaactcttgtaaattgaagtatgaaaatatactgaattgggtattatatgaatttttctgatttaggattcacatgatccaaaaaggaaa

SEQ ID NO: 26 - DNA, 10891 bp, artificial
  Vector comprising artificial microRNA precursor
gtgattttgtgccgagctgccggtcggggagctgttggctggctggtggcaggatatattgtggtgtaaacaaattgacgcttagacaacttaataacacattgcggacgtctttaatgtactgaatttagttactgatcactgattaagtactgatatcggtaccaattcgaatccaaaaattacggatatgaatataggcatatccgtatccgaattatccgtttgacagctagcaacgattgtacaattgcttctttaaaaaaggaagaaagaaagaaagaaaagaatcaacatcagcgttaacaaacggccccgttacggcccaaacggtcatatagagtaacggcgttaagcgttgaaagactcctatcgaaatacgtaaccgcaaacgtgtcatagtcagatcccctcttccttcaccgcctcaaacacaaaaataatcttctacagcctatatatacaacccccccttctatctctcctttctcacaattcatcatctttctttctctacccccaattttaagaaatcctctcttctcctcttcattttcaaggtaaatctctctctctctctctctctctgttattccttgttttaattaggtatgtattattgctagtttgttaatctgcttatcttatgtatgccttatgtgaatatctttatcttgttcatctcatccgtttagaagctataaatttgttgatttgactgtgtatctacacgtggttatgtttatatctaatcagatatgaatttcttcatattgttgcgtttgtgtgtaccaatccgaaatcgttgatttttttcatttaatcgtgtagctaattgtacgtatacatatggatctacgtatcaattgttcatctgtttgtgtttgtatgtatacagatctgaaaacatcacttctctcatctgattgtgttgttacatacatagatatagatctgttatatcattttttttattaattgtgtatatatatatgtgcatagatctggattacatgattgtgattatttacatgattttgttatttacgtatgtatatatgtagatctggactttttggagttgttgacttgattgtatttgtgtgtgtatatgtgtgttctgatcttgatatgttatgtatgtgcagctgaaccatggcggcggcaacaacaacaacaacaacatcttcttcgatctccttctccaccaaaccatctccttcctcctccaaatcaccattaccaatctccagattctccctcccattctccctaaaccccaacaaatcatcctcctcctcccgccgccgcggtatcaaatccagctctccctcctccatctccgccgtgctcaacacaaccaccaatgtcacaaccactccctctccaaccaaacctaccaaacccgaaacattcatctcccgattcgctccagatcaaccccgcaaaggcgctgatatcctcgtcgaagctttagaacgtcaaggcgtagaaaccgtattcgcttaccctggaggtacatcaatggagattcaccaagccttaacccgctcttcctcaatccgtaacgtccttcctcgtcacgaacaaggaggtgtattcgcagcagaaggatacgctcgatcctcaggtaaaccaggtatctgtatagccacttcaggtcccggagctacaaatctcgttagcggattagccgatgcgttgttagatagtgttcctcttgtagcaatcacaggacaagtccctcgtcgtatgattggtacagatgcgtttcaagagactccgattgttgaggtaacgcgttcgattacgaagcataactatcttgtgatggatgttgaagatatccctaggattattgaggaagctttctttttagctacttctggtagacctggacctgttttggttgatgttcctaaagatattcaacaacagcttgcgattcctaattgggaacaggctatgagattacctggttatatgtctaggatgcctaaacctccggaagattctcatttggagcagattgttaggttgatttctgagtctaagaagcctgtgttgtatgttggtggtggttgtttgaattctagcgatgaattgggtaggtttgttgagcttacggggatccctgttgcgagtacgttgatggggctgggatcttatccttgtgatgatgagttgtcgttacatatgcttggaatgcatgggactgtgtatgcaaattacgctgtggagcatagtgatttgttgttggcgtttggggtaaggtttgatgatcgtgtcacgggtaagcttgaggcttttgctagtagggctaagattgttcatattgatattgactcggctgagattgggaagaataagactcctcatgtgtctgtgtgtggtgatgttaagctggctttgcaagggatgaataaggttcttgagaaccgagcggaggagcttaagcttgattttggagtttggaggaatgagttgaacgtacagaaacagaagtttccgttgagctttaagacgtttggggaagctattcctccacagtatgcgattaaggtccttgatgagttgactgatggaaaagccataataagtactggtgtcgggcaacatcaaatgtgggcggcgcagttctacaattacaagaaaccaaggcagtggctatcatcaggaggccttggagctatgggatttggacttcctgctgcgattggagcgtctgttgctaaccctgatgcgatagttgtggatattgacggagatggaagctttataatgaatgtgcaagagctagccactattcgtgtagagaatcttccagtgaaggtacttttattaaacaaccagcatcttggcatggttatgcaatgggaagatcggttctacaaagctaaccgagctcacacatttctcggggatccggctcaggaggacgagatattcccgaacatgttgctgtttgcagcagcttgcgggattccagcggcgagggtgacaaagaaagcagatctccgagaagctattcagacaatgctggatacaccaggaccttacctgttggatgtgatttgtccgcaccaagaacatgtgttgccgatgatcccgaatggtggcactttcaacgatgtcataacggaaggagatggccggattaaatactgagagatgaaaccggtgattatcagaaccttttatggtctttgtatgcatatggtaaaaaaacttagtttgcaatttcctgtttgttttggtaatttgagtttcttttagttgttgatctgcctgctttttggtttacgtcagactactactgctgttgttgtttggtttcctttctttcattttataaataaataatccggttcggtttactccttgtgactggctcagtttggttattgcgaaatgcgaatggtaaattgagtaattgaaattcgttattagggttctaagctgttttaacagtcactgggttaatatctctcgaatcttgcatggaaaatgctcttaccattggtttttaattgaaatgtgctcatatgggccgtggtttccaaattaaataaaactacgatgtcatcgagaagtaaaatcaactgtgtccacattatcagttttgtgtatacgatgaaatagggtaattcaaaatctagcttgatatgccttttggttcattttaaccttctgtaaacattttttcagattttgaacaagtaaatccaaaaaaaaaaaaaaaaaatctcaactcaacactaaattattttaatgtataaaagatgcttaaaacatttggcttaaaagaaagaagctaaaaacatagagaactcttgtaaattgaagtatgaaaatatactgaattgggtattatatgaatttttctgatttaggattcacatgatccaaaaaggaaatccagaagcactaatcagacattggaagtaggaatatttcaaaaagttttttttttttaagtaagtgacaaaagcttttaaaaaatagaaaagaaactagtattaaagttgtaaatttaataaacaaaagaaattttttatattttttcatttctttttccagcatgaggttatgatggcaggatgtggatttcatttttttccttttgatagccttttaattgatctattataattgacgaaaaaatattagttaattatagatatattttaggtagtattagcaatttacacttccaaaagactatgtaagttgtaaatatgatgcgttgatctcttcatcattcaatggttagtcaaaaaaataaaagcttaactagtaaactaaagtagtcaaaaattgtactttagtttaaaatattacatgaataatccaaaacgacatttatgtgaaacaaaaacaatatctagtacgcgtcaattgatttaaatttaattaatcgactctagattcgacggtatcgataagctcgcggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccctgaaagcgacgttggatgttaacatctacaaattgccttttcttatcgaccatgtacgtaagcgcttacgtttttggtggacccttgaggaaactggtagctgttgtgggcctgtggtctcaagatggatcattaatttccaccttcacctacgatggggggcatcgcaccggtgagtaatattgtacggctaagagcgaatttggcctgtaggatccgcgagctggtcaatcccattgcttttgaagcagctcaacattgatctctttctcgatcgagggagatttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcgaggcgcgcctctcaaactcgtttgtgctgagagaaccaagggtttcttcccttgcagagaagaaaatttggatacatatggttaagtttatttagatatcttgtttgttctttcatctttctcagatatggtttcgaaatgggagattggggatgacgtcttctatctgaccatacagaaaaagctttaaaggttggattttgaggctatccctttatgtgatctggatggtcagaaagaagacgtcaaccttatctctctctcttgagatcttctcccatggaggggtgatggcttatgtatataggtttcccagctgtagcatctttagggtttgagattcctaaccatctttacttcctgtcctgcaggcctaggatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcggccggccgtttaaacttagttactaatcagtgatcagattgtcgtttcccgccttcactttaaactatcagtgtttgacaggatatattggcgggtaaacctaagagaaaagagcgtttattagaataatcggatatttaaaagggcgtgaaaaggtttatccgttcgtccatttgtatgtcaatattgggggggggggaaagccacgttgtgtctcaaaatctctgatgttacattgcacaagataaaaatatatcatcatgaacaataaaactgtctgcttacataaacagtaatacaaggggtgttcgccaccatgagccatatccagcgtgaaacctcgtgctcccgcccgcgcctcaattccaatatggatgccgacctttatggctacaagtgggcgcgcgacaacgtcggccagtcgggcgcgaccatttatcggctttatggcaaacccgatgccccggaactgttcctgaagcacggcaaaggcagcgtcgcaaacgatgtcaccgatgagatggtccgcctgaactggcttaccgagttcatgccgctgccgacgattaagcatttcatccgtaccccggacgatgcctggctcttgaccacggccattccgggcaaaacggcctttcaggtccttgaagagtacccggactccggtgagaatatcgtggacgccctcgcggtcttcctccgccgtttgcatagcatccccgtgtgcaactgccccttcaactcggaccgggttttccgcctggcacaggcccagtcgcgcatgaataacggcctcgttgacgcgagcgatttcgacgatgaacggaatggctggccggtggaacaggtttggaaggaaatgcacaaactgcttccgttctcgccggattcggtggtcacgcatggtgatttttccctggataatctgatctttgacgagggcaagctgatcggctgcatcgacgtgggtcgcgtcggtatcgccgaccgctatcaggacctggcgatcttgtggaattgcctcggcgagttctcgccctcgctccagaagcgcctgttccagaagtacggcatcgacaacccggatatgaacaagctccagttccacctcatgctggacgaatttttttgaacagaattggttaattggttgtaacactggcagagcattacgctgacttgacgggacggcggctttgttgaataaatcgaacttttgctgagttgaaggatcgatgagttgaaggaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcataggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccggggtcgatcaaaaagtaatcggggtgaaccgtcagcacgtccgggttcttgccttctgtgatctcgcggtacatccaatcagcaagctcgatctcgatgtactccggccgcccggtttcgctctttacgatcttgtagcggctaatcaaggcttcaccctcggataccgtcaccaggcggccgttcttggccttcttggtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggcggggcctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccccttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgctcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccaaatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagatgatca